(19) United States
(12) Patent Application Publication
(10) Pub. No.: US 2013/0081484 A1
(43) Pub. Date: Apr. 4, 2013

(54) GAS CHROMATOGRAPHY INLET LINERS AND SAMPLE PATH CONTAINERS

(71) Applicants: Brian A. Jones, Bellefonte, PA (US); Scott L. Grossman, Bellefonte, PA (US); Valerie S. Strohm, Bellefonte, PA (US); Thomas E. Kane, Port Matilda, PA (US)

(72) Inventors: Brian A. Jones, Bellefonte, PA (US); Scott L. Grossman, Bellefonte, PA (US); Valerie S. Strohm, Bellefonte, PA (US); Thomas E. Kane, Port Matilda, PA (US)

(21) Appl. No.: 13/516,359

(22) PCT Filed: Dec. 15, 2010
(86) PCT No.: PCT/US10/03175

§ 371 Date: Dec. 3, 2012

Related U.S. Application Data
(60) Provisional application No. 61/284,231, filed on Dec. 15, 2009.

(51) Int. Cl. G01N 33/00; B05D 1/18; G01K 13/00

(52) U.S. Cl. CPC G01N 33/00; G01K 13/00; B05D 1/18
USPC 73/864.91; 73/865.8; 374/141; 427/430.1; 427/157; 427/160

(57) ABSTRACT

A container used for chromatographic sample analysis has (a) at least one silica uppermost layer, which is deposited onto the container using a liquid phase deposition process, and, optionally, (b) at least one indicator present in at least part of the at least one silica uppermost layer for visually distinguishing the container from other containers, and/or determining the “use status” of the container, and/or identifying the proper orientation of the container when it is to be put in use, and/or providing a means of decoration, identification, or counterfeit detection. In one preferred embodiment, the indicator is thermochromic. In another preferred embodiment, the indicator is photochromic.

### BACKGROUND OF THE INVENTION

Gas chromatography (GC) is a well known analytical technique where gas phase mixtures are separated into their individual components and subsequently identified. The technique may be employed to obtain both qualitative and quantitative information about the components of the mixture. Specifically, the separation mechanism employs two different media, one moving (mobile phase) and one unmoving (stationary phase). In GC, the mobile phase is normally hydrogen or helium, which flows across the stationary phase, which is a solid or otherwise immobilized liquid on a solid support on the interior capillary wall.

The sample mixture is introduced into the mobile phase stream and the residence time of each component of the sample in the stationary phase relates to differences in their individual partitioning constants with respect to the two solvent phases.

Samples for GC are usually liquid and must be volatized prior to introduction to the mobile phase gas stream. GC analysis is typically divided into five stages:
1. Sample storage, where analytical samples (including standards) are managed prior to insertion to the instrument,
2. sample preparation, where samples are inserted into the instrument, heated, and (if in liquid form) volatilized,
3. sample loading, where the sample vapor is loaded all or in part onto the analytical column,
4. separation, where the sample is separated into its individual components as it passes through the analytical column, and
5. detection, where the separated components are identified as they exit the analytical column.

In conventional GC instrumentation, step 1 usually involves glass vials or containers, often loaded into an autosampler device. In this fashion the autosampler may contain multiple samples in vials slated for analysis. Steps 2 and 3 are achieved in the sample inlet hardware. Inlet hardware often includes a sample injection syringe and a replaceable sleeve, or liner. Liners are normally operated at elevated temperatures, e.g., over 200′C. This enhances the rate of sample vaporization and reduces adsorption on the inner surface of the liner [1]. Many internal configurations are available for liners, as well as coatings for them [2-12].

In most cases the configuration of the liner serves to enhance the degree of sample volatilization from the point of exit from the syringe needle to the column entrance, and provide gas phase sample homogeneity from components within the liquid mixture having different boiling points. A simple configuration for an inlet liner is a straight cylindrical tube of glass having a consistent inner diameter along the longitudinal path. Other configurations include more complex inner paths intended to increase turbulence, affect the comparatively short residence time the liquid sample is in the liner, or interrupt the liquid stream leaving the syringe needle. These internal configurations include tapers or goosenecks, baffles, funnels, inverted cup elements, spiral regions, points of flow constriction, and drilled holes along the longitudinal path of the liner.

Other elements of liners optionally include small plugs of packing materials such as glass wool [1], Carbofrit™ (Trademark of Restek Corporation) packing material, and more recently inert metal wire bundles [13] which serve as additional surface area sources for heat transfer into the sample and as a physical filter for any solid/nonvolatile contaminants present in the liquid sample.

During the injection process, it is also important to minimize sample/liner and (if applicable) sample/packing material interactions that can result in undesirable chemical reactions, decomposition, or temporary/permanent adsorption of the sample, It is equally important that the liner not contribute contaminants to the analysis, which may result in spurious peaks or an increase in the baseline signal in detection measurements of the components contained in the sample being analyzed.

In cases where liners become contaminated with solid/nonvolatile species it is necessary to replace them from time to time. Liner replacement frequency depends on the type of sample; with ‘dirty’ samples having high concentrations of high boiling point components or large amounts of nonvolatile particulate matrix shortening the service life of the liner.

Because of the techniques commonly employed in using a liner in a GC instrument it is often desirable for the liner to be transparent. It is particularly important to be able to see through the walls of these liners which contain packing material in order to ensure its proper plug position within the internal bore of the liner and to be able to check for the presence of debris or other visual contaminants.

Various chemical coatings are applied to liners in order to reduce the degree of interaction between the sample and the surface of the liner. Sample-surface interactions may result in sample absorption in the coatings, decomposition of the coatings, and formation of new reaction products; in each case resulting in undesirable peaks (or loss of desirable ones) in detection measurements of the components contained in the sample being analyzed in the separation analysis. In addition to low sample-surface interactions, it is also desirable for the liner coating to be thermally stable in order to minimize background signal contributions originating from the liner coating itself detected by the analytical equipment. For glass substrate liners, common deactivation techniques include chemically treating the exposed silanol groups with organosilane reagents such as hexamethyldisilazane (HMDS), dimethyldichlorosilane (DMCS), and trimethylchlorosilane (TMCS) [15].

Liners are manufactured from glass, primarily borosilicate, but also fused quartz, and less commonly from metal, mainly stainless steel [16].

The chemical composition of a quartz or fused silica liner is commonly 99.4-99.9% silicon dioxide (SiO₂). The uppermost surface of the SiO₂ silica contains a uniform, high concentration of silanol (Si—OH) groups. This represents the optimal surface with which to apply the deactivation chemistry described. An even distribution of silanol groups across the silica surface ensures a uniform deactivation coating which creates the most effective sample barrier [1].

While quartz is chemically the preferred material to make liners, the high melting point temperature (1650° C.) required to manipulate the cylindrical shape makes quartz practical strictly for straight tube liner designs (See Table 1)[17]. The difficulty in manipulating fused quartz into precision shapes has led to the need for subsequent machining or grinding operations on the stock tubing. While the initially drawn tubing is smooth and free of non-silica defects, these features are lost in subsequent processing. The risks of impurity introduction by machining or grinding, as well as the introduction of scratches and crevices, complicate production of optimal fused quartz liners for GC use.

Similar limitations are observed for sample vials manufactured from quartz. Quartz vials must be manufactured at temperatures higher than that required for borosilicate glass. Vials manufactured from quartz requires higher temperature furnaces and tooling having higher heat resistance. This greatly increases the costs for quartz vials as compared to those made from borosilicate glass or other glasses having comparatively lower softening points.

Laboratory borosilicate glass begins to soften at ca. 650° C., which enables comparatively low temperature thermoforming with which to create the complex shapes like those depicted in FIGS. 1 and 2A-2I. Pyrex borosilicate glass includes a relatively high concentration of non-silica moieties (ca.19%) including primarily boron, sodium, aluminum, and potassium. To create a pure silica surface wherein pure is defined as containing at least 90% wt. silica and hydroxylated silica, these contaminating species are generally removed by aqueous acid leaching prior to application of deactivation coatings [1]. The general commercial manufacturing stages for fused silica and borosilicate glass liners are described in FIGS. 3a and 3b. For the case of borosilicate glass, the surface purity following acid leaching resembles that of virgin fused quartz (or fused silica) with the advantage of the bulk item having the low temperature thermoforming ability of the borosilicate glass substrate.

Acid leaching of the borosilicate glass article has been shown to leave a roughened silica enriched surface by solubilizing and removing the accessible domains enriched with non-silica components. The surface resembles that of silica gel following this leaching process with superficial pores commonly around 5 nanometers in average diameter, but which may be smaller or much larger for some specialty borosilicate glasses [18-23]. Because of sterile limitations within the thus-formed pores and crevices, a number of silanol groups are unreachable for coverage by the silane deactivation reagent. A lack of coverage completeness of the deactivation coating yields a population of residual active sites capable of interaction with analytes in the finished article. [1] There is a need in the field of gas chromatography for easily formable containers fabricated from glass and with a very low surface area pure silica surface suitable for effective chemical deactivation with traditional reagents.

In addition to silane treatments, another suitable deactivation coating is vapor deposited hydrogenated amorphous silicon, including the Siltek® Sulfinert® coatings [17, 24-26]. These coatings have been demonstrated on both borosilicate glass and metal surfaces, and in both cases it results in a semi-opaque mirror finish whose color is dependant on the coating thickness.

While chemically inert to many analytes, the Sulfinert® surface is not represented as being perfectly smooth. The creation of the amorphous hydrogenated silicon first layer takes place through decomposition of silane gas, and a layer of silicon may not completely cover the entire substrate surface after one silicon deposition cycle. The silicon deposition cycle is usually repeated several times to build up the passive layer of silicon to the requisite thickness. [25]. The surface morphology of these coatings has been studied and SEM data shows the presence of a textured surface for Silcosteel®, a related coating [27].

### Discussion on Liquid Phase Deposition (LPD)

Liquid phase deposition of a silica film on glass sheets from fluorosilicic acid solutions under mild conditions was first described by Kawahara [28]. It was developed as an alternative to more costly sputtering and chemical vapor deposition techniques for large objects, and was shown to give a more dense silica coating than that obtained through selective leaching processes. The motivation for this work was to be able to economically alter the light reflecting properties of large glass sheets, as well as reduce alkali metal migration for battery, liquid crystal display, and solar cell applications, as well as to provide an electrically insulating layer for electronic devices.

The reversible reactions can be exploited to deposit, precipitate, and/or change composition through evaporative or distillative processes depending on choice of conditions. The equilibria of mixtures of fluorosilicic acid and silica have been explored in detail [29-33].

H₂SiF₆+2H₂O⇄SiO₂↓⇄+6HF

5H₂SiF₆+SiO₂↓⇄3[H₂SiF₆.SiF₄]+2H₂O⇄3H₂SiF₆SiF₄↑+2H₂O

It is clear that several components are present in equilibrium in an aqueous solution of fluorosilicic acid. As these can vary in relative concentration, we refer to any stable, homogeneous liquid reaction product of silica, hydrogen fluoride and water in this invention as fluorosilicic acid. In the event that a change occurs which renders the homogeneous solution unstable with regard towards precipitation of solid silica in any form, the solution shall be referred to as being supersaturated with silica.

It is apparent from the equilibria described above that through increasing the relative concentration of a component through addition, or by reducing one by depletion, the balance can shift to either precipitate or dissolve silica [29, 34-85]. For example, addition of water to a silica saturated fluorosilicic acid solution renders it supersaturated, leading to solid silica precipitation or its deposition as a film [29, 68-85].

Much of the development done on silica based LPD has dealt with deposition on silicon and glass sheets. Silicon substrates have been extensively used with LPD in semiconductor manufacturing as a means of producing conformal insulating films with low dielectric constants. Film densities, impurity incorporation, the effect of annealing conditions and their associated influence on electrical behavior have been extensively studied. Studies involving deposition on glass have generally centered on altering visual properties, such as reflectivity or color.

Deposition of silica from fluorosilicic acid solutions is always accompanied by some level of fluoride incorporation in the film. A typical range is 1.8-6.25 wt. % [62, 74]. Conditions producing higher film growth rates produce higher fluoride concentrations. This is not just a surface phenomenon; rather the fluorine is resident throughout the film as treatment with boiling water has been demonstrated to have a limited effect on reducing bulk fluoride concentration [65].

Removal of fluorine following LPD from fluorosilicic acid solutions is possible at elevated temperatures. Thermal annealing is effective in facilitation of fluoride migration to the surface, where it is lost from the film to the gas phase. With treatment at ≧700° C. the fluorine is completely removed from the surface. While these conditions are acceptable to silica layers deposited on silicon substrates, these temperatures are sufficiently high so as to physically alter or deform the targeted dimensions of borosilicate substrates and should therefore be avoided. Because of this intolerance of high temperatures, a non-zero weight percent fluoride content on the surface of the borosilicate glass remains and can be used as an effective indicator that glass articles have been subjected to a LPD coating process employing fluorosilicic acid solutions. It is our opinion that the introduction of permanent fluoride contamination into LPD treated glass substrates may have previously served as a deterrent toward consideration of this type of coating by others skilled in the art for articles destined for use in the flow path of a chromatographic system.

The annealing induced outward migration of fluoride is accompanied by a densification of the film and reduction of porosity to levels approaching that of thermally deposited silica [74]. Annealing also reduces film stress values from those produced during the deposition process. Auger electron spectroscopy has been used to determine atomic composition depth profiles of LPD deposited and annealed coatings [44, 47, 71]. The thickness and refractive index of the LPD silica layer can be determined by ellipsometry [47, 57, 61, 71, 81, 83].

LPD based silica films from a saturated fluorosilicic acid solution have been demonstrated to yield smoother surfaces having a lower surface area per square unit of space as compared to untreated glass substrates. The technology is employed in the solid state electronics industry to planarize and smooth silicon-based substrates [35, 39].

### Vials/Vessels for Analytical Samples

In the same way it is important to minimize sample/liner interactions that can result in undesirable chemical reactions, decomposition, or permanent adsorption of the sample, it is equally important that the glass vessels used to store the sample prior to introduction to the GC inlet also have the same inert qualities. Common vessels employed in GC analysis to store samples include glass vials (generally for on-site prepared samples) [17] and ampoules (generally for sample standards or long term storage) [17]. Sample vials are commonly available made of borosilicate glass [17], less commonly quartz and much less commonly metal. Sample vials and ampoules are commonly used to contain samples in gas as well as liquid form. In the interest to further protect the chemical integrity of the samples being stored, often the vials are chemically deactivated using similar procedures as described for borosilicate liners [17].

Because the sample is at least temporarily contained in the liner until it is introduced into the GC column, the liner device itself can be described as a sample container within the GC sample path. For the purpose of this disclosure the term ‘container’ is used to describe any device employed to store or direct the sample throughout the GC analytical path and includes glass or metal sample vials, ampoules, cylinders, inlet liners, GC columns, or coupling devices employed to direct the sample to the GC detector.

### Discussion on Doping and Dopants

Organic and organometallic compounds with useful optical and electronic properties have been used as dopants in porous Vycor glass [92-94].

Organic dyes have been infused into Vycor pores [95-97]. Unblocked pores containing dopants have been used in gas sensing applications [98]. The dopants have also been encapsulated by organic polymers within these pores [99-103].

The mild conditions required for the creation of sol-gel glasses is compatible with a wide range of dopants that constitute the same family as those described above for infusion into porous Vycor glass. Because of a lack of interconnected pores, the chromophoric species are usually present during the gelation process. Further ripening of the gel can take place without damaging the entrained dyes, salts, or chromophoric particles [104-110].

### Discussion on Thermochromic and Photochromic Dyes

Dyes that produce a color change with temperature are referred to as thermochromic and have been the subject of extensive study [111-117]. A number of chemical classes exhibiting this behavior have been reported, and include perylene dyes, encapsulated leucodyes, and some inorganic compounds.

In addition, many photochromic compounds (those that change color on exposure to light at specific wavelengths) exhibit thermochromic behavior. Some examples of this class of compounds include spiropyrans, spiroxazines, and ethylene aromatics, and the color change is due to often reversible molecular rearrangements giving rise to differences in conjugation or ionic structure. Thermochromic and photochromic indicators have been introduced into both porous Vycor glass, and sol-gel glasses, with the polarity and geometric constraints in their vicinity affecting the stabilization of one isomeric form over another. Thus environmental effects can result in thermochromic or reverse thermochromic behavior with the same dye molecule. Encapsulation of the dyes within the pores of these inorganic glass matrices has been reported using polymethylmethactylate to yield transparent composites with the anticipation that they could lead to the development of three-dimensional high density memory arrays [108].

The thermochromic effect can be gradual, with the color intensity varying with the percentage of (lye molecules in each state. Photochromic images previously set through light exposure can sometimes be erased through thermochromic relaxation.

### SUMMARY OF THE INVENTION

We present here a borosilicate-based liner or container before deactivation having a smooth, high purity silica uppermost surface, where the uppermost surface is deposited using a liquid phase deposition process, where high purity silica is defined as having less than a total of 10% of a combination of boron oxides, and metal or other element oxides besides silicon on a mass basis. We thus disclose a superior process to standard leaching technologies as the leaching process leaves behind a comparatively high surface area, high surface energy substrate, which can compromise the completeness and uniformity of the final liner or container's deactivation coating.

We also describe a method of annealing that is adequate to remove contaminating fluoride concentrations to acceptable levels so as not to interfere with subsequent deactivation processes. We also define the uppermost surface of the liner or container as the region extending inward from zero to 100 nanometers from the surface of the article. For the purpose of this disclosure the uppermost layer may result from multiple repeat LPD cycles on the same substrate. The term ‘layer’ and ‘uppermost layer’ may refer to at least one layer deposited on the substrate surface, with more than one layer being present next to or on top of one another.

We further present the application of LPD based silica films, with a subsequent deactivation process, with glass containers used for handling or storing chemical samples.

Previously, liners employing color indicators have been demonstrated using porous glass substrates [14]. Similar practice has been demonstrated here where the indicator is incorporated into the LPD layer, which can be deposited on both porous and non-porous containers [86-91].

We further present a borosilicate liner or container having a smooth, high purity silica uppermost surface, where the uppermost surface incorporates an indicator.

We further present a borosilicate liner or container having a smooth, high purity silica uppermost surface, where the uppermost surface incorporates multiple overlaying layers where at least one of the layers incorporates an indicator.

We further present a liner or container having a deposited high purity silica layer, which employs indicators within the silica layer as a means to easily identify one liner or container from another. For the purpose of this disclosure the term indicator relates to any element, compound, dopant, or mixture additive to the glass substrate that modifies the native color or transparency of the glass substrate.

We further present here a liner or container having a deposited high purity silica layer which employs indicators as a means of visually identifying the thermal state of the liner.

We further present here a liner or container having a deposited high purity silica layer which employs indicators as a means of determining “use status”, wherein it is possible to visually identify if the liner or container has been previously subjected to high temperature and optionally the duration of exposure beyond a specific threshold.

We further present here a liner or container having a deposited high purity silica layer which employs indicators with controlled physical placement as a means of identifying the orientation of the liner.

We further present here a liner or container having a deposited high purity silica layer which employs colored indicators that are visible under ordinary and/or ultraviolet light as a means of decoration, identification, or counterfeit detection.

In addition, we present a liner or container having a deposited high purity silica layer wherein the indicators are effectively blocked from surface interactions through thermally stable chemical capping.

We further present a liner or container having a deposited high purity silica layer which employs indicators receptive to photolithographic imaging.

### BRIEF DESCRIPTION OF THE DRAWINGS

FIG. 1 are representations of several types of sample vials employed for storing and distributing analytical samples and sample standards.

FIGS. 2A through 2I are representations of several types of gas chromatography injection port liners known in the art.

FIG. 3a represents the general manufacturing process (Scheme 1) for quartz liners, FIG. 3b represents the general manufacturing process (Scheme 2) for borosilicate liners or containers employing the acid leach process, and FIG. 3c represents the inventive manufacturing process (Scheme 3) for borosilicate liners or containers of the invention employing the LPD process.

FIGS. 4 through 10 are representations of various aspects of the preferred embodiment of a liner utilizing our invention that interfaces into a gas chromatograph injection port. A similar composition or structure may also be inferred for other types of containers. The enlarged portions of the figures represent close-up views of the inner and outer walls of the liners or containers, showing the various layers and coatings deposited thereon.

FIG. 11 is a graphical representation of a chromatogram generated of a chemical mixture injected directly onto a column, bypassing contact with an injection port liner using a deactivated injection port liner constructed according to the prior art and illustrated in FIG. 1.

FIGS. 12 through 17 are graphical representations of chromatograms generated of various chemical mixtures using both commercially available injection port liners (FIGS. 12, 13, 15, and 16) and prototypes (FIGS. 14 and 17) constructed according to the preferred embodiments of this invention.

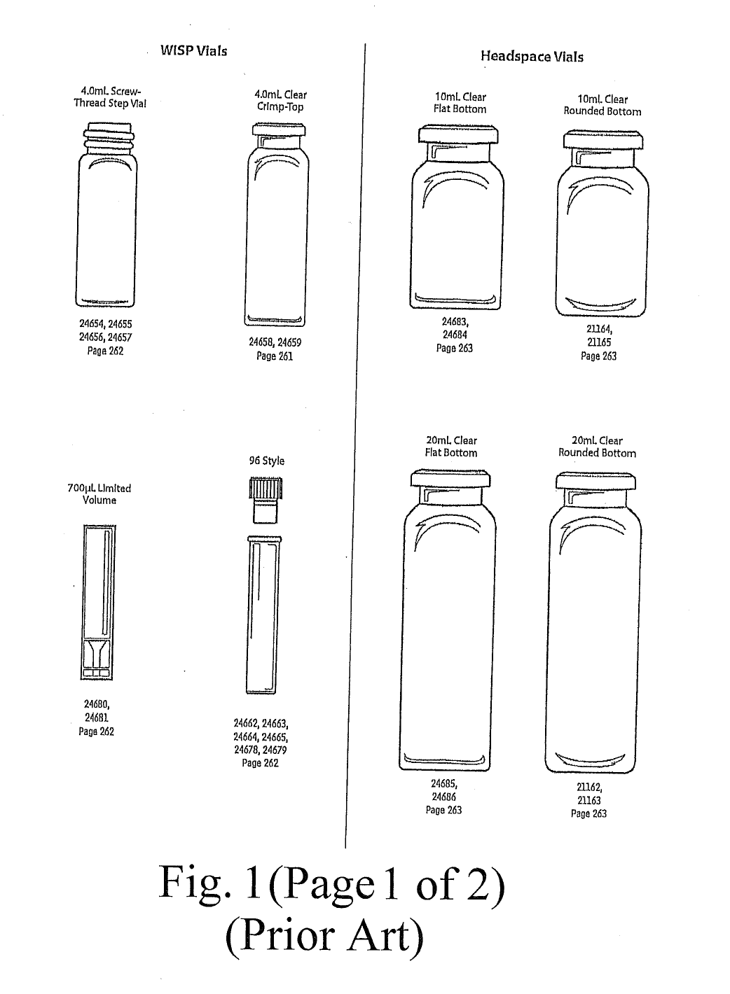

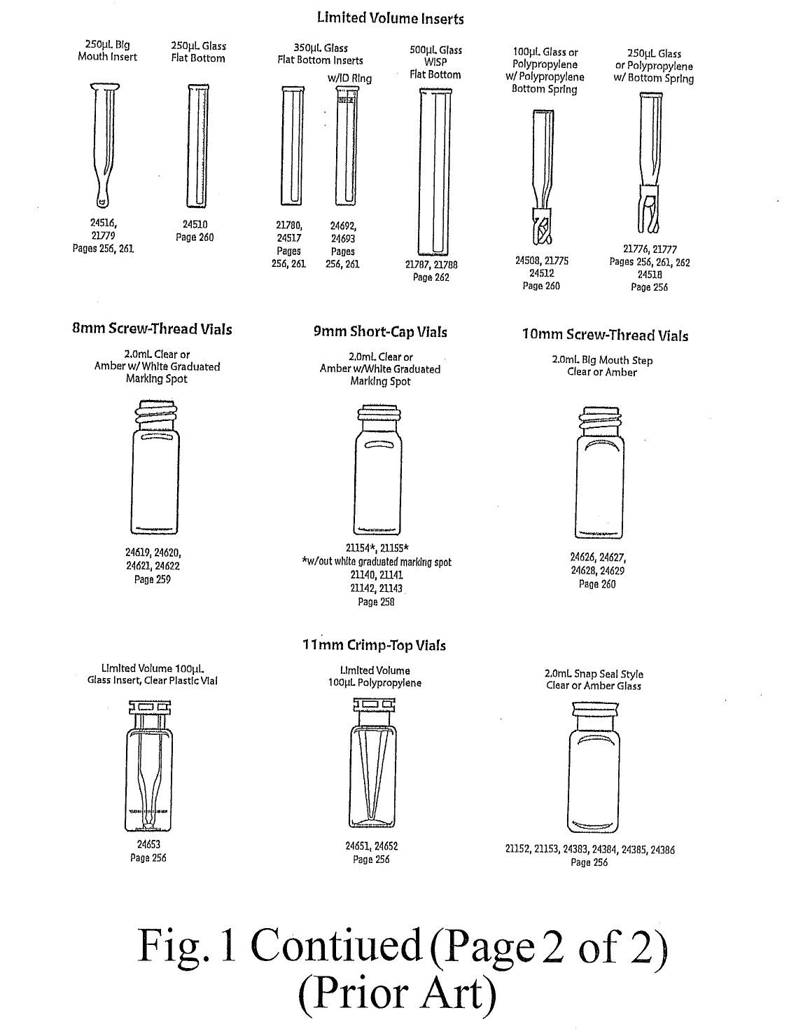

### DETAILED DESCRIPTION OF THE PREFERRED EMBODIMENTS

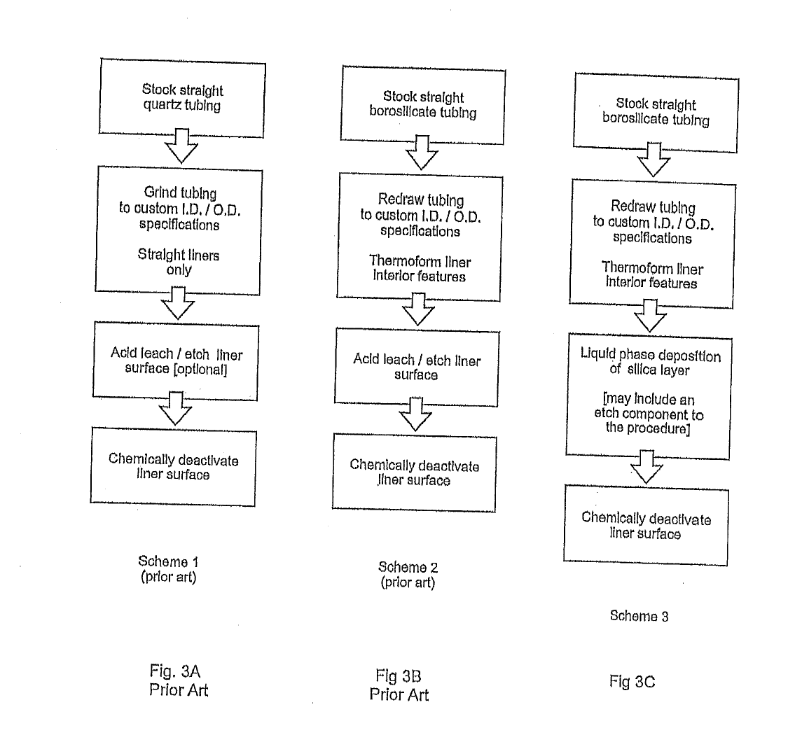

In one preferred embodiment of this invention as illustrated in FIG. 3c, a container such as a borosilicate liner or vial is leached with diluted hydrochloric acid to remove the accessible boric and alkali metal oxides, and then washed to neutrality with deionized water. The article is then immersed in a silica saturated (or supersaturated) solution of fluorosilicic acid, optionally containing additives or indicators so as to impart the desired properties in the finished product. The temperature may optionally be raised to accelerate the silica deposition rate. At the conclusion of treatment, the article is removed from the solution and rinsed thoroughly with deionized water. It is then placed in a vessel and while purged with an inert gas, it is heated to as high as 550° C. from several minutes to several hours before cooling.

The resulting liner or container is thus coated with a film exhibiting the properties shown in Table 1, and is finally deactivated with silanes using procedures described in the art.

TABLE 1
Properties of glass compositions

| | | Unleached | |
|---|---|---|---|
| | Unleached | Fused Silica/ | LPD coating (uppermost |
| | Borosilicates | Quartz | surface layer) |
| Impurities | ca. 15-30% | <1.0% | <10% |
| (boron oxide | | | |
| and metal oxides | | | |
| basis), wt. | | | |
| Fluorine, % wt. | ~0.00 | ~0.00 | Between 0.05 and 10% |
| SiO2, wt. | ca. 70-85% | balance | balance |
| Softening | ca. 650-820 | ca. 1650 | does not significantly |
| point, ° C. | | | contribute to softening |
| | | | point of the substrate |
| Annealing | ca. 560 | ca. 1210 | does not significantly |
| point, ° C. | | | contribute to annealing |
| | | | point of the substrate |

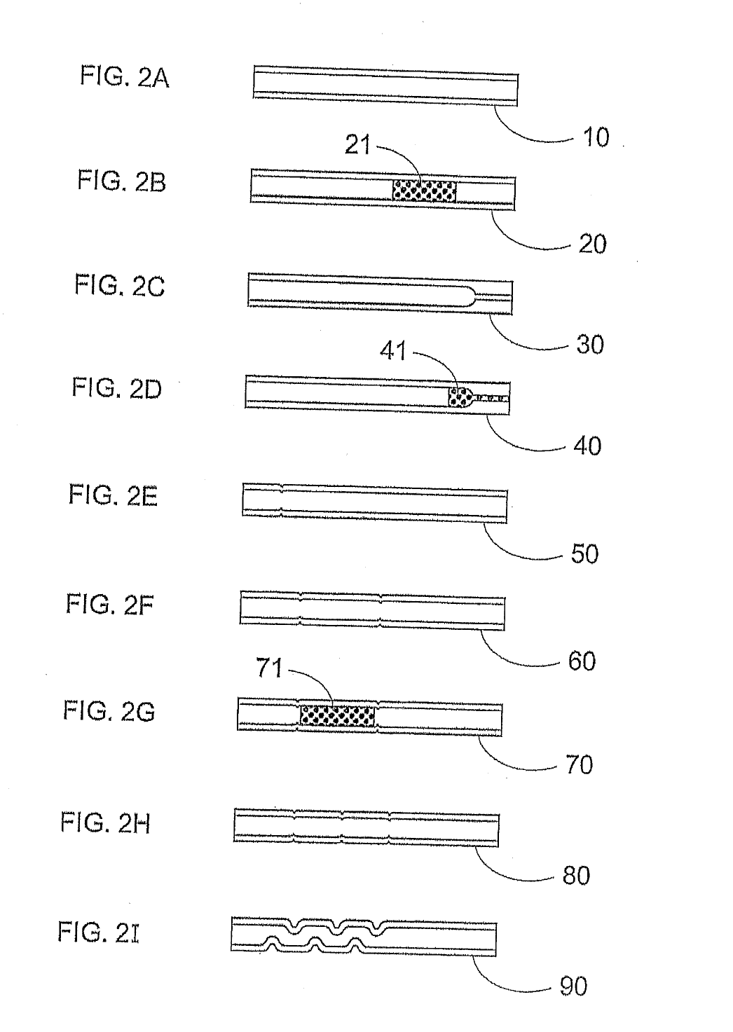

In FIGS. 2A-2I, sectional views of various sample inlet liner configurations known in the art are illustrated. FIG. 2A is an example of a straight through sample inlet liner 10. FIG. 2B illustrates a liner 20, comprising the liner 10 incorporating a matrix 21, which may be comprised of wool, particles, wire bundles, or other materials know in the art, FIG. 2C illustrates a single taper liner configuration 30, FIG. 2D illustrates a liner 40 having the same configuration as in liner 30, with the addition of a matrix 41, similar in composition to that designated as 21 in FIG. 2B. FIG. 2E illustrates a sample inlet liner 50 with dimples or constrictions extending into the inner bore. FIG. 2F illustrates a liner configuration 60 with two sets of internal dimples or constrictions; and FIG. 2G represents the same type of liner, with the same two sets of dimples or constrictions 70, but with the addition of a matrix 71 described above that is supported within the zone created by said dimples or constrictions. FIG. 2H illustrates a liner 80 having a straight through sample inlet liner with yet three sets of internal dimples or constrictions; and FIG. 2I illustrates a sample inlet liner 90 with multiple sets of interior baffles.

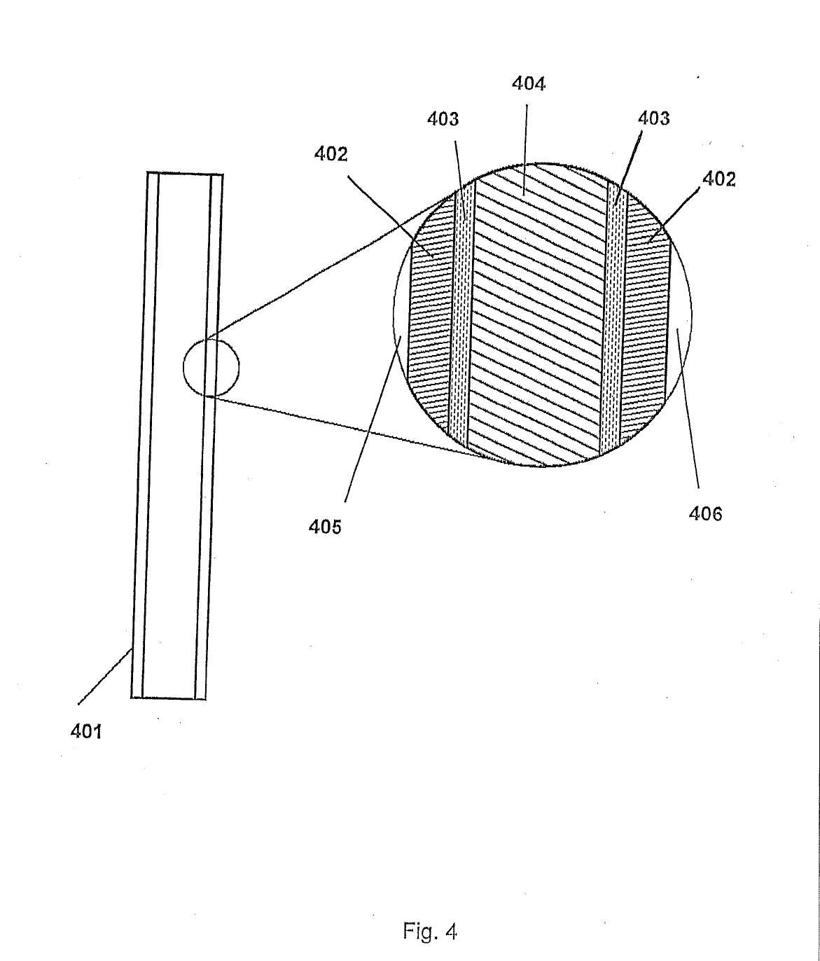

In FIG. 4, 401 is an example of the present invention and is fabricated from borosilicate glass. In the enlarged view of a portion of this figure, 405 represents the inner region of the liner tube, with 406 being the outer. Region 404 represents the borosilicate glass body from which the liner is fabricated, with 403 illustrating a thin film of silicon dioxide deposited on the inner and outer walls using the process of this invention. In order to eliminate absorptive activity towards analytes, the liner is coated with a final layer of a deactivant that is usually silane or siloxane in nature, but may be comprised of other moieties as known in the art, and designated as 402.

FIG. 5 is a rendering of a borosilicate or quartz liner with the main body shown as 501, and the wall view expanded as 505 and the inner and outer regions designated 508 and 509, respectively. Section 504 represents a film of silicon dioxide deposited on the glass surface using the LPD process and containing one or more types of dye molecules 506 co-deposited at the same time, and fully encapsulated within the film. The liner is coated inside and out with a deactivant as in FIG. 2 and designated in this Figure as 502.

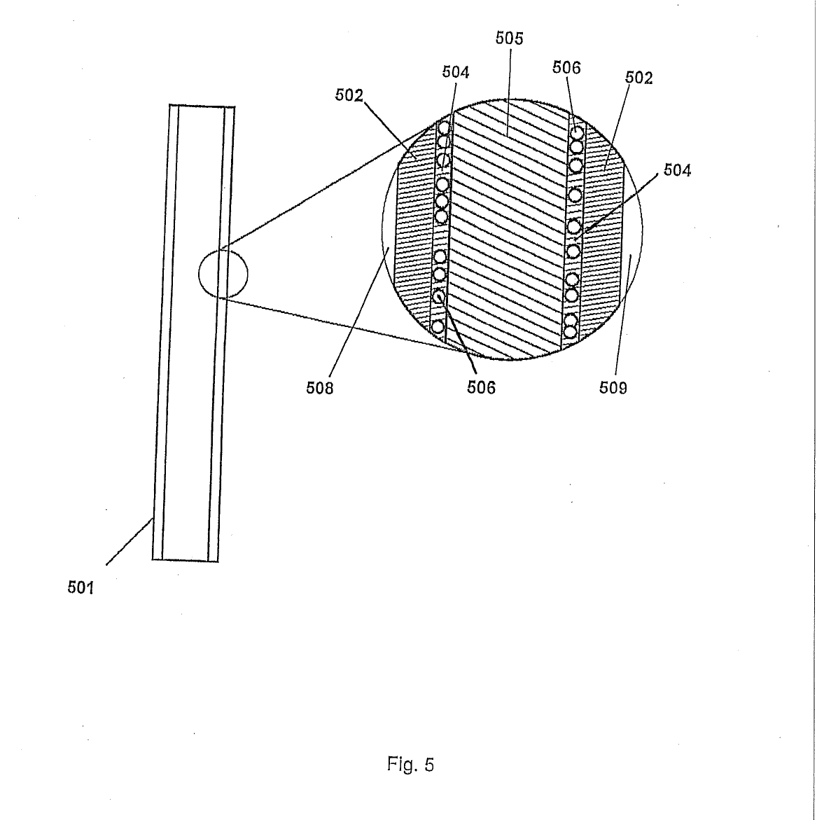

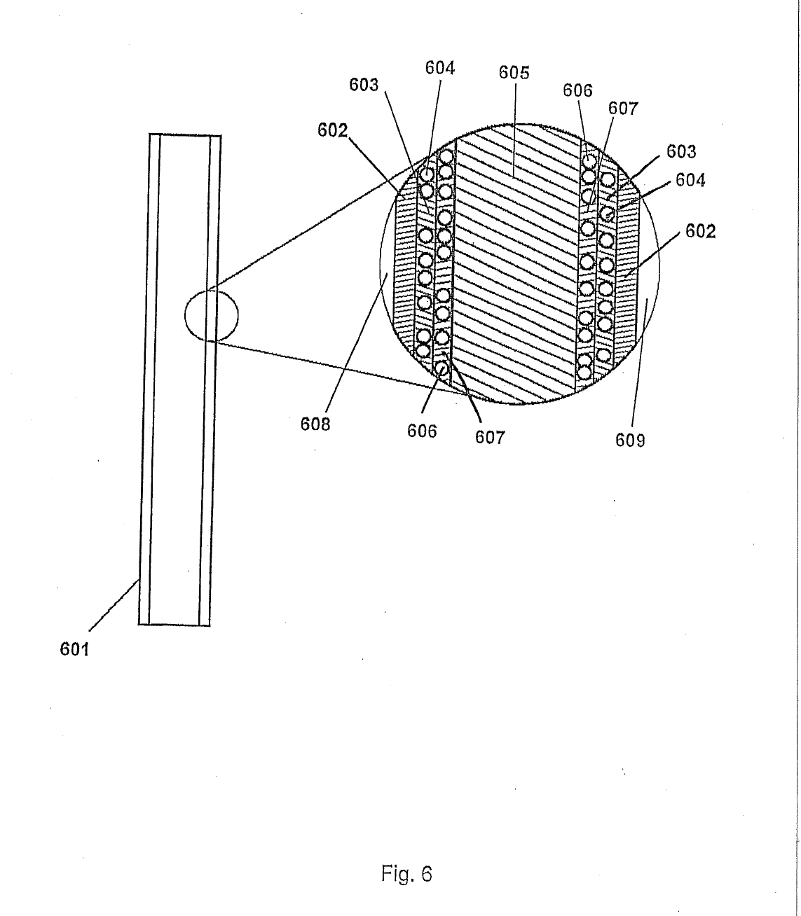

FIG. 6 illustrates a borosilicate or quartz liner 601 as in the preceding Figure, and expanded as 605, but with the addition of a second silica deposition layer 603 containing one or more dye molecule families (604) that may be the same or different from those (606) deposited in the first silica layer 607. Again, the inner and outer wall regions are designated in this figure as 608 and 609, respectively. The liner is coated inside and out with a deactivant as in the preceding. Figure and designated in this Figure as 602.

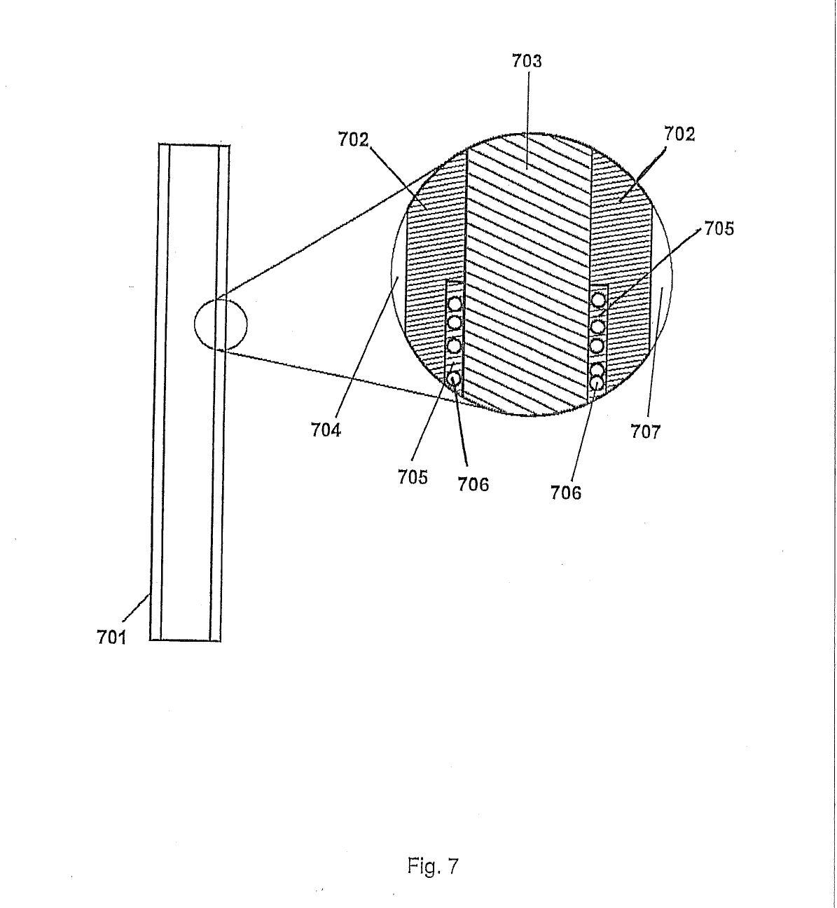

The liner represented in FIG. 7 as 701 and the core glass or quartz wall designated as 703, contains a selected region that is coated with a silica film 705 and containing one or more dye molecule families 706 co-deposited and fully encapsulated within the film. Selected deposition may be accomplished by partial immersion into a silica saturated fluorosilicic acid solution containing the desired dye(s) or by masking off of regions where solution contact is to be prevented. Again, a suitable deactivant film 702 is shown applied on the inner 704 and outer 707 walls of the liner to minimize analyte interaction with residual active sites.

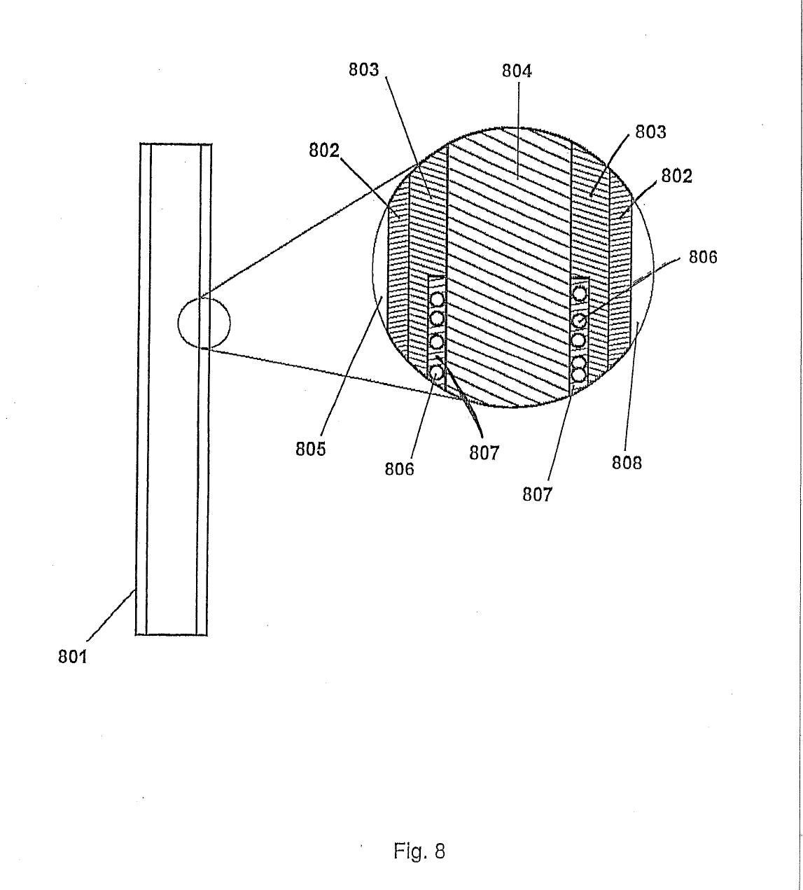

The liner illustrated in FIG. 8 and represented by 801 and body 804 contains a layer of liquid phase deposited silica 807 in a selected region only and optionally containing one or more types of dye molecules 806. This layer is shown on both inside 805 and outside 808 surfaces of the tube, but may be selectively placed on only one, or with myriad gaps as desired according to the masking/immersion method used. The LPD layer first placed on the tube and the remaining surfaces are coated with a second silica film using the methods of this invention 803 that totally encapsulates the liner. In this Figure, the second coating of silica is clear and is intended to illustrate a barrier layer capable of barring passage or diffusion of any buried atoms. A deactivant layer 802 is shown as a final coating to mask silanol sites or other residual reactive regions.

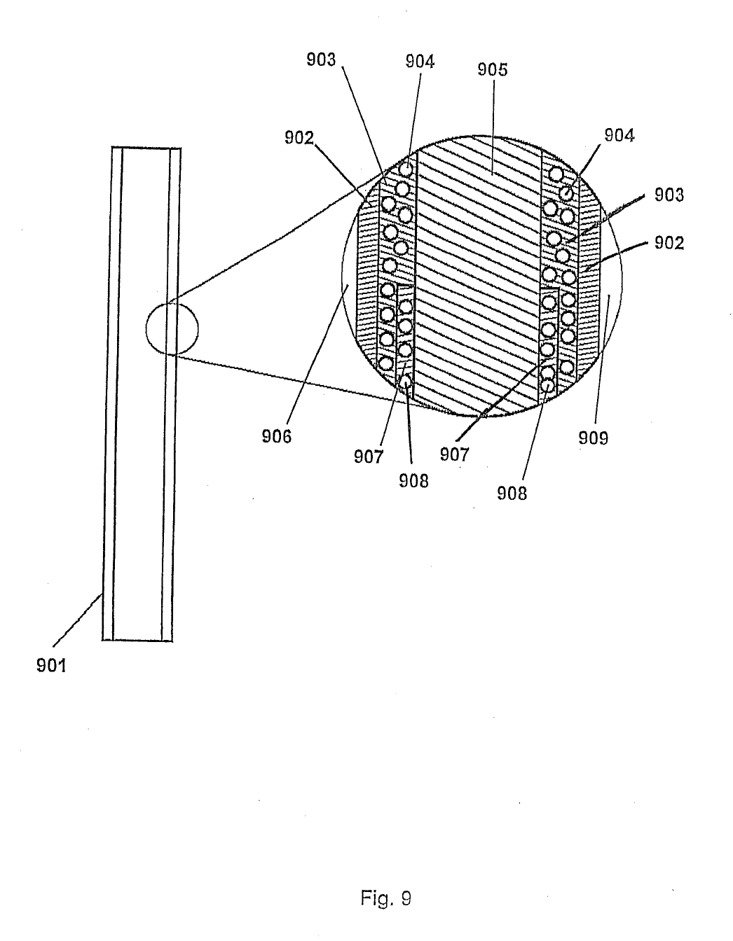

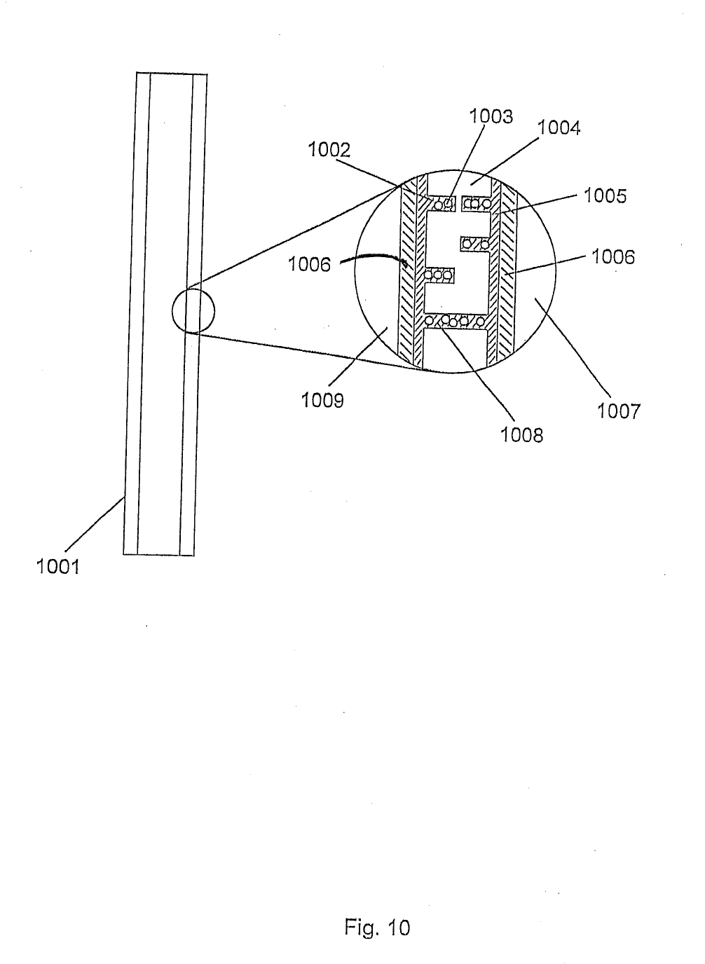

The liner illustrated in FIG. 9 and represented by 901 and body 905 contains a layer of liquid phase deposited silica 907 in a selected region only and optionally containing one or more types of dye molecules 908. This layer is shown on both inside 906 and outside 909 surfaces of the tube, but may be selectively placed on only one, or with myriad gaps as desired according to the masking/immersion method used. The LPD layer first placed on the tube and the remaining surfaces are coated with a second silica film using the methods of this invention 903 that totally encapsulates the liner. In this Figure, the second coating of silica also contains one or more families of dye molecules 904 intended to both shield the underlying surface from exposure to the environment and impart a secondary visual effect. A deactivant layer 902 is shown as a final coating to mask silanol sites or other residual reactive regions. The tube in FIG. 10 as illustrated as 1001 is a representation of another aspect of the embodiment of this invention, with an expanded view as 1004. The region 1008 shows the presence of “through pores”, or pores providing fluidic communication between the inner 1009 and outer 1007 walls of the liner, such as would be found with porous Vycor glass as the substrate material. These pores are not initially present in the tube, but result from an acid leaching process wherein interconnected regions of borate-rich glass that permeate through the glass are selectively solubilized and removed. The section identified as 1002 is a superficial pore that extends inward from either the interior 1009 or exterior 1007 surface of the tube. This type of pore exists in Vycor type glasses, but they also are prevalent on the surface of acid leached borosilicate glasses (including Pyrex and other brands) that have not undergone phase separation into continuous regions. These pores are generally small and don't penetrate more than a few tenths of microns from the surface, but their physical presence is easily noted as an increased surface roughness after acid leaching compared to that of the initial glass. All pores contribute to an increased surface area over that of the solid body [18-23]. The present illustration shows the presence of a film of LPD silica that fills both superficial and through-hole pores and covers the exposed surfaces of the tube. It optionally can contain one or more families of dye molecules 1003 that are encapsulated within the clear film 1005 to impart the desired optical properties to the object. This film also accomplishes a hole-plugging task that effectively eliminates fluidic communication between the inner and outer surfaces of the tube. The liner may be deactivated with a suitable film shown as 1006 to provide inertness toward analytes.

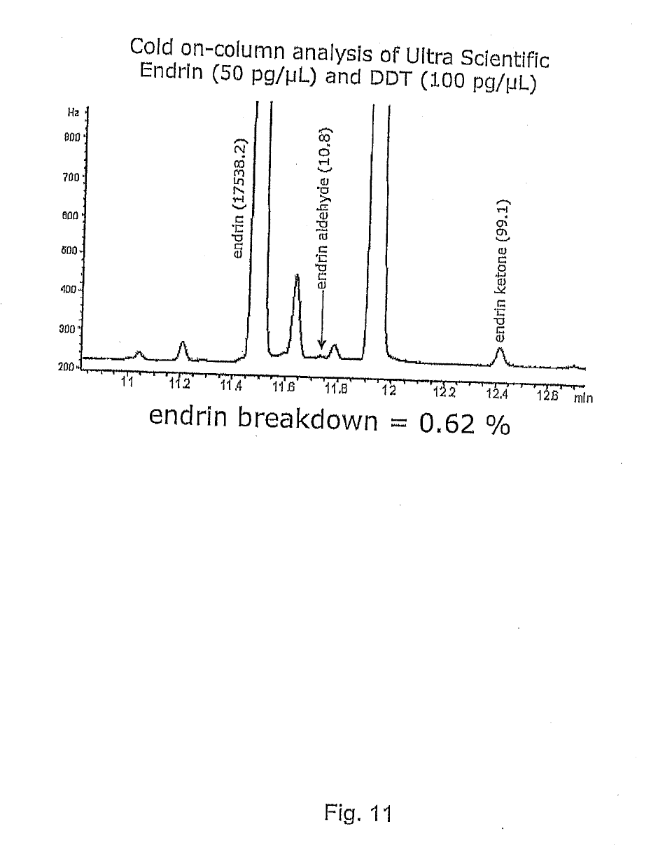

FIG. 11 depicts a section of a chromatogram of a pesticide mixture that was obtained from Ultra Scientific. The key areas illustrated show the elution region of endrin and its typical breakdown products endrin aldehyde and endrin ketone. It also shows the presence of DDT, another chlorinated pesticide. The chromatogram was acquired using cold on-column injection of 1 μL to eliminate injection port liner contributions to analyte breakdown, and used a 15 meter, 0.53 mm ID, 1 um film, Rxi-5MS column for the separation. The run conditions were: Inlet: 50° C. (hold 0.5 min) to 280° C. @ 20° C./min (hold 3 min), Constant flow @ 5 mL/min (He); Oven: 45° C. (hold 0.5 min) to 275° C. @ 20° C./min (hold 3 min); Detector: uECD, 310° C., N₂ make-up gas flow of 60 mL/min.

The peaks are identified on the chromatogram with their absolute area counts in parentheses, and the bulk endrin and DDT concentrations in the test mixture were 50 and 100 pg/μL respectively. The identification of the eluted peaks and their absolute area counts was based on previous studies with internal standards. In this experiment, the endrin breakdown was found to be 0.62%, which may be considered a baseline value for this particular standard using the separation column and conditions indicated.

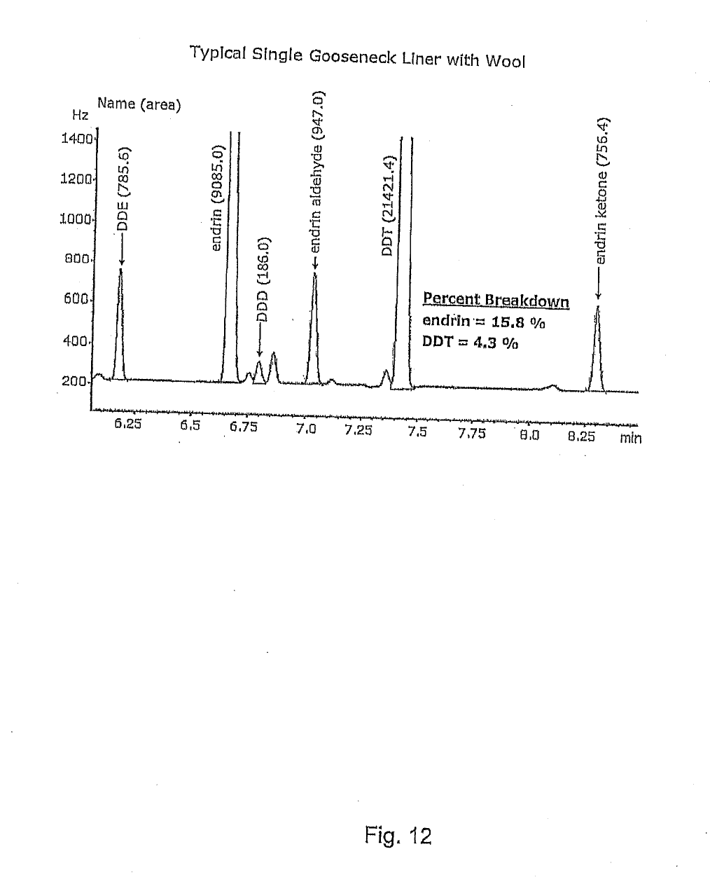

FIG. 12 illustrates a chromatogram of a commercial single gooseneck style liner containing borosilicate wool, and manufactured according to typical procedures reported in the art, with the analytical conditions listed below and using the mixture in FIG. 11:

Inlet temperature: 250° C., Splitless injection, at 20 mL/min @ 0.5 min, constant flow @ 1.5 mL/min, helium carrier

### Oven: 150° C. to 240° C. @ 20 C/min to 265° C. @ 6° C./min

Column: 20 meter, 0.18 mm ID, 0.18 μm film, Rxi-5sil-MS
Detector: μECD, 300° C., N₂ make-up gas flow of 60 mL/min

The same analytical standard was used for the analysis, and 1 μL was injected. The endrin breakdown level was found to be 15.8% and that of DDT to be 4.3%. The difference in breakdown levels from those found in FIG. 11 may be attributed to analyte breakdown in the heated injection port liner.

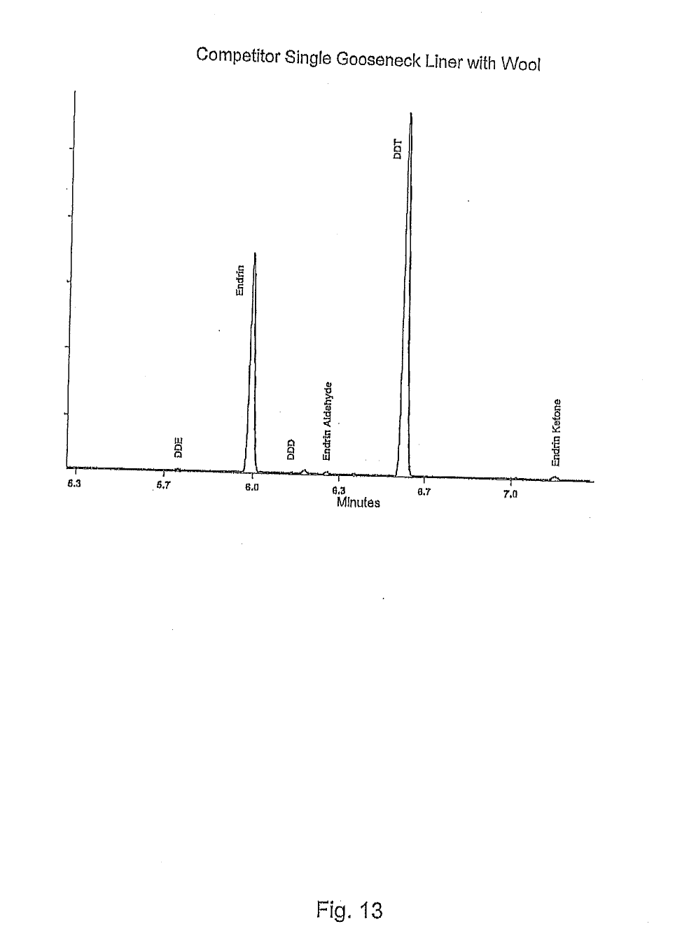

FIG. 13 shows a chromatogram using the best competitive liner available as determined by exhaustive benchmark testing. It was tested using the mixture from FIG. 11 with the following conditions:

Inlet temperature: 220° C., Splitless injection, 15 sec. hold time, 20 mL/min purge flow, column flow @ 1.5 mL/min, helium carrier
Oven: 120° C. (hold 1 min.) to 225° C. @ 20° C./min to 280° C. @ 6° C./min. (hold 7 min)
Column: 20 meter, 0.18 mm ID, 0.18 um film, Rxi-5-MS
Detector: μECD, 290° C., N₂ make-up gas flow of 60 mL/min

The endrin breakdown level was found to be 2.8% and that of DDT to be 1.2%. The difference in breakdown levels from those found in FIG. 11 may be attributed to analyte breakdown in the heated injection port liner.

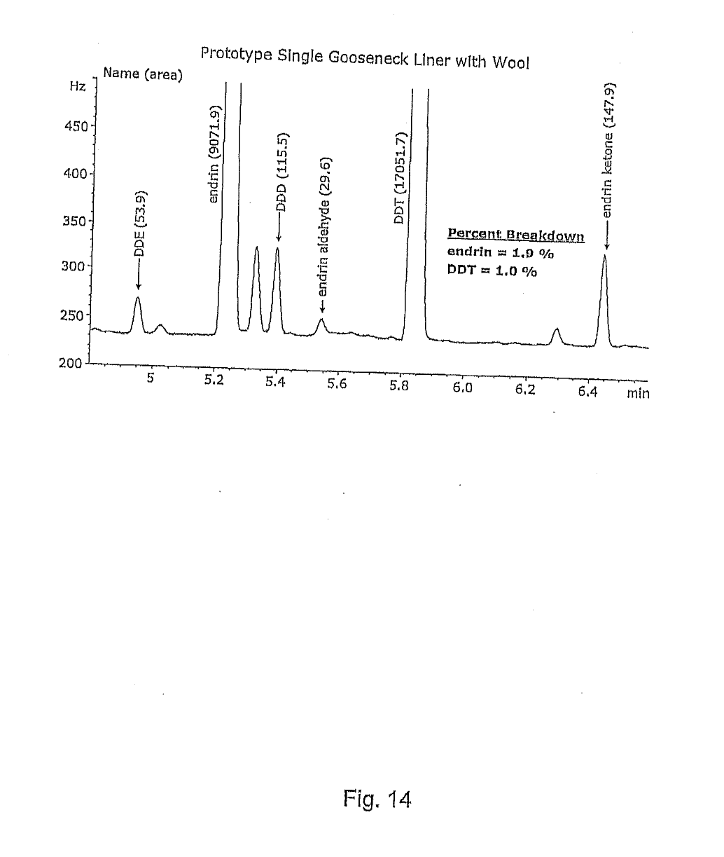

FIG. 14 illustrates a chromatogram obtained in the analysis on the pesticide mixture in FIG. 11 with an injection port liner fabricated with a liquid phase deposition layer of silica containing Rhodamine B dye below the deactivation layer, and prepared according to this invention. The analytical conditions for this run were the same as FIG. 12.

The endrin breakdown level was found to be 1.9% and that of DDT to be 1.0%. The difference in breakdown levels from those found in FIG. 11 may be attributed to analyte breakdown in the heated injection port liner. It should be noted that this improved inertness toward analyte breakdown was achieved with the prototype liner of the invention compared to the prior art liner used in connection with FIG. 13, even though the temperature of the injection port was 30° C. higher here than in the prior case (FIG. 13). Increasing the injection port temperature is known to increase the amount of endrin and DDT breakdown in an analysis.

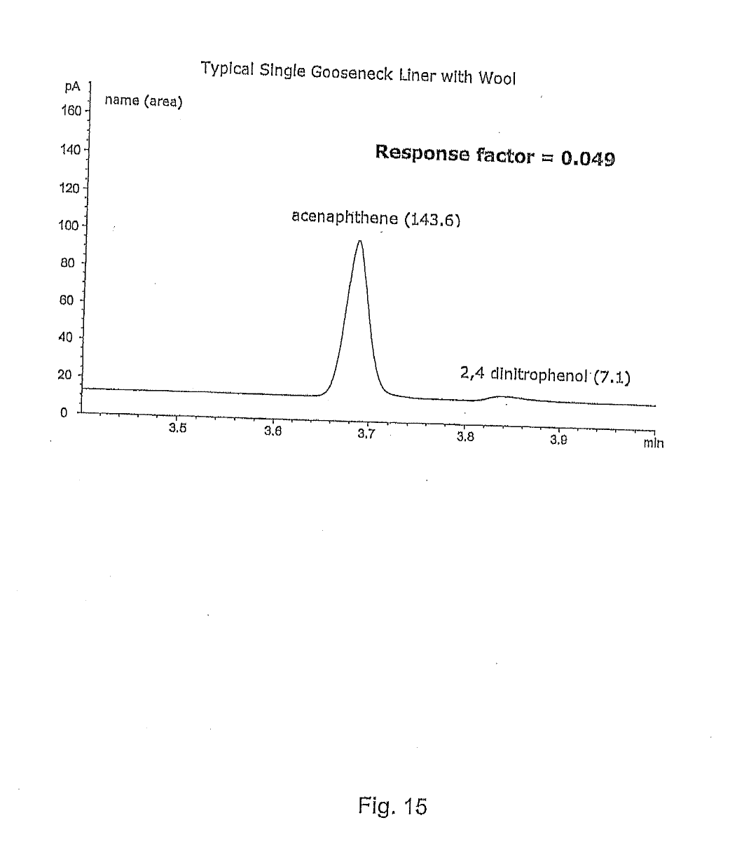

The chromatogram depicted in FIG. 15 was generated to quantitate a typical level of loss of 2,4-dinitrophenol, which is an acidic analyte shown to be sensitive toward active sites in GC injection port liners. Its response is reported relative to the internal standard acenaphthene using cold on-column injection analysis, and the value indicating no absorptive losses is 0.31. This single gooseneck liner containing wool gave a response factor of 0.049, when run under the following conditions:

Inlet: 250° C., Constant pressure at 50 psi, Splitless injection, split flow @ 20 mL/min @ 1 min

### Oven: 50° C. to 150° C. @ 20° C./min

Column: 15 m, 0.25 mm ID, 0.25 um film, Rxi-5MS
Detector: FID, 300° C., H₂ @ 40 mL/min, Air @ 450 mL/min, N₂ @ 45 mL/min
The sample was 1 μl, of a 10 ng/μL solution of 2,4-dinitrophenol and 10 ng/μL of acenaphthene

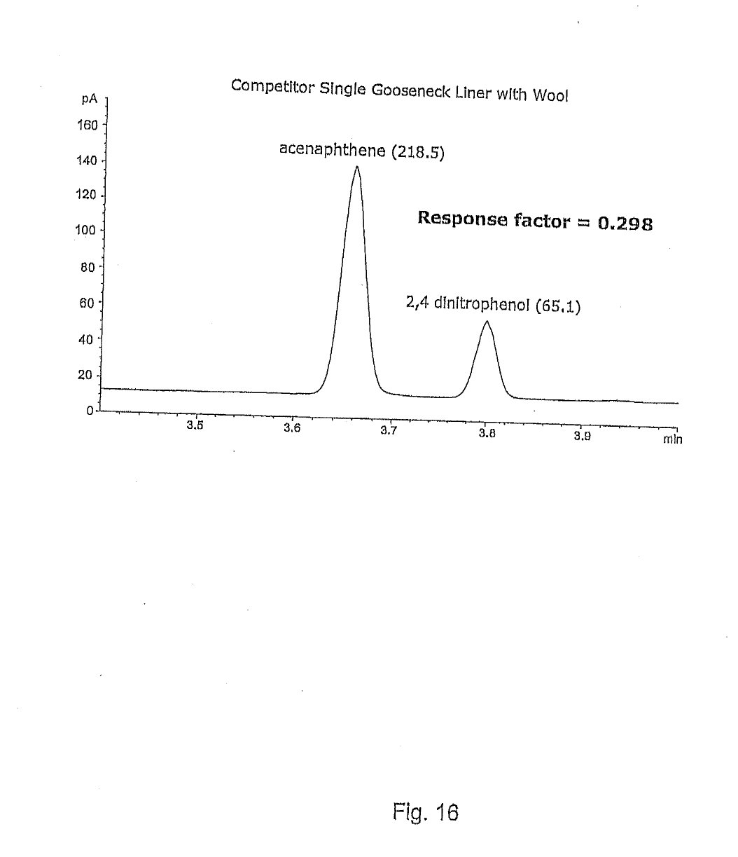

FIG. 16 shows a chromatogram using the best competitive liner available as determined by exhaustive benchmark testing for the analysis of 2,4-dinitrophenol. The conditions and the sample were the same as in FIG. 15, and the response factor for 2,4-dinitrophenol was 0.298 relative to acenaphthene.

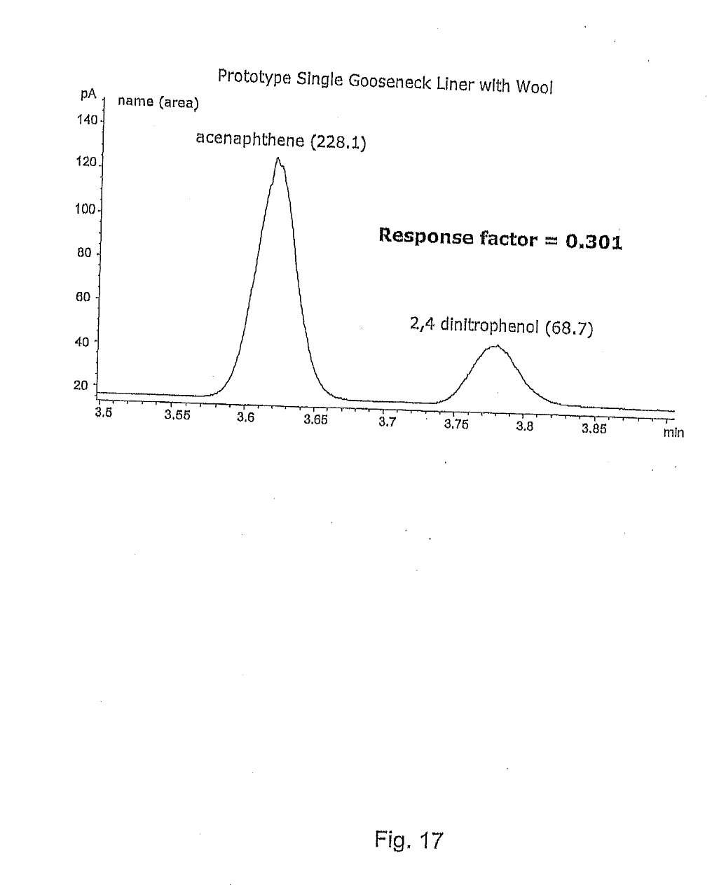

FIG. 17 illustrates a chromatogram obtained in the analysis of 2,4-dinitrophenol with an injection port liner of the invention fabricated with a liquid phase deposition layer of silica containing Rhodamine B dye below the deactivation layer, and prepared according to this invention. The conditions and the sample were the same as in FIG. 15, and the response factor for 2,4-dinitrophenol was, 0.301 relative to acenaphthene, showing at least comparable results to those in FIG. 16, and a considerable improvement over the results with the liner tested in FIG. 15.

### EXPERIMENTAL RESULTS

The following examples are presented to show various aspects of the process of the invention.

### Example 1

In this example, simple coloration of a single gooseneck borosilicate glass liner, illustrated in FIG. 2C is described. Silica powder was added in intervals to a 3.2M solution of fluorosilicic acid and stirred magnetically until no more dissolved. The suspension was then placed in a freezer at −9° C. overnight. A portion of the suspension was taken and diluted 1:1 with a 0.2% solution of Rhodamine Bin water. After brief mixing, the suspended silica was removed by filtration through a 0.45 μm PTFE filter. A 4 mm ID single gooseneck borosilicate glass liner suitable for use in an Agilent 6890 Gas Chromatograph was rinsed with acetone, dried, and then immersed in the freshly filtered solution. The vessel was capped, and periodically mixed. After 4 hours, the liner was removed from the solution and rinsed well with water. It exhibited a transparent dark pink/purple color that was uniform throughout.

### Example 2

A non-cationic dye was utilized as a colored indicator, even though it has limited water solubility in its acid form, but still exhibiting an intense fluorescence. A silica saturated fluorosilicic acid (SSFA) solution prepared as in Example 1 above was filtered and diluted with an equal volume of an aqueous solution saturated with fluorescein. A liner as in Example 1 was immersed in the solution and agitated periodically over 16 hours. The liner was removed from the solution, rinsed well with water and dried. It appeared almost colorless, but exhibited some visible fluorescence when illuminated with ultraviolet light.

### Example 3

A SSFA solution prepared as in Example 1 above was filtered and diluted and mixed with an equal volume of a 0.2% solution of Crystal Violet dye in water. Crystal Violet is a member of the family of triphenylmethane cationic dyes, whose color is a function of the local pH. A liner was treated with this solution as in Example 2. It exhibited a transparent light blue color. The liner was rinsed well with water until neutral pH was achieved and no dye was extractable from the glass surface. The liner was then dried in a forced air oven. After overnight treatment at 200° C., the color was as before, with no visible change having occurred.

### Example 4

A liner was treated as in Example 3 above, but with Malachite Green oxalate (another cationic dye from the triphenylmethane family) as the dye. It exhibited a transparent dark green color. The liner was rinsed well with water until neutral pH was achieved and no dye was extractable from the glass surface. The liner was then dried in a forced air oven. After overnight treatment at 200° C., the color was as before, with no visible change having occurred.

### Example 5

Examination of less thermally stable dyes as thermochromic indicators was demonstrated in this example. A borosilicate glass liner was treated as in Example 3 above, but with the phenothiazine compound Methylene Blue hydrate as the dye. It exhibited a transparent dark blue color. The liner was rinsed well with water until neutral pH was achieved and no blue dye was extractable from the glass surface. The liner was then dried in a forced air oven. After overnight treatment at 200° C., the color was gone, leaving the liner clear and colorless.

### Example 6

A liner was treated as in Example 3 above, but with anionic Methyl Orange as the dye. It showed no visible evidence of dye incorporation. This result is consistent with reports in the literature that cationic dyes are more likely to be incorporated in the growing silica film than anionic ones.

### Example 7

A liner was treated as in Example 3 above, but with another anionic compound, Trypan Blue as the dye. It showed, no visible evidence of dye incorporation. This result is consistent with reports in the literature that cationic dyes are more likely to be incorporated in the growing silica film than anionic ones.

### Example 8

Dyes with higher thermal stability and thermochromic behavior as indicators were utilized in the following example. A borosilicate glass liner was treated as in Example 3 above, but with Neutral Red as the dye. It exhibited a transparent brick red color. The liner was rinsed well with water until neutral pH was achieved and no dye was extractable from the glass surface. The liner was then dried in a forced air oven. After overnight treatment at 200° C. in air, the color was as before, with no visible change having occurred. The temperature was increased to 400° C. and the liner was examined after 1 hour exposure. It had turned a transparent light brown color. Heating was continued at 400° C. in air and the liner periodically examined for color change. None occurred and the experiment was terminated after 72 hours accumulated heating time at 400° C.

### Example 9

The effect of the LPD process with wool present inside the liner, along with annealing and deactivation were demonstrated in the following example. A single gooseneck borosilicate glass liner was loaded with 6 mg of 4 μm (nominal fiber diameter) fused quartz wool and leached with 3M HCl solution for 30 minutes. The acid was removed by rinsing with water until the pH was neutral. The liner was then oven dried at 120° C. for 30 minutes. The initially smooth glass surface was noticeably rougher after the acid leaching process as determined by physical feel.

Silica saturated fluorosilicic acid solution prepared in Example 1 was allowed to warm to room temperature and was held for 24 hours. The excess silica was removed by filtration and 20 mL of this solution was placed in a plastic tube. The solution was diluted with 20 mL of water that had 2 drops of a 0.2% Rhodamine B solution added to it. After mixing well, the acid leached liner was immersed in the solution and allowed to stand 6 hours with occasional swirling. The liner was then removed from the solution and rinsed with water until the pH was neutral. A faint pink transparent coloration of the glass was observed, with more intensity of color on the wool surface. The rough surface had also developed a very smooth feel as a result of the deposition treatment. The liner was placed in a stainless steel vessel, purged with helium, and heated to 250° C. for 5 hours to anneal the freshly deposited silica and reduce the overall fluoride content. The liner was then deactivated using standard reagents and processes and evaluated for inertness by gas chromatography. Results are shown in FIGS. 14 and 17.

### Example 10

A SSFA solution prepared as in Example 1 above was filtered, diluted and mixed with an equal volume of water. Two drops of Rhodamine B solution (0.2% in water) was added, and a single gooseneck liner was immersed in the solution and the plastic vessel containing it was closed. The vessel was placed in an oven at 35° C. and within 2 hours pink particles had formed and coated both the inside of the vessel and the liner. The liner itself showed a pink coloration similar to the liner from Example 9 after water rinsing and drying, but with a significant haze. The surface of the liner was also much rougher in feel than the liner from Example 9. This example demonstrated the effect of combined dilution and heating in one step. Homogeneous nucleation occurred from the supersaturation level being too high, such that particle formation competed with film growth.

### Example 11

In this example, the LPD solution was not diluted with an equal volume of water, but was heated in order to effect silica supersaturation. A SSFA solution prepared as in Example 1 above was filtered and mixed with two drops of Rhodamine B solution (0.2% in water). A single gooseneck liner was immersed in the solution in a closed plastic vessel. The vessel was placed in an oven at 35° C. and allowed to heat overnight. The vessel was cooled and the liner rinsed well with water until pH neutral. The liner was transparent with a transparent light pink color and exhibited no sign of haziness, indicating that only film formation occurred without homogeneous nucleation. The liner surface was shiny and smooth.

### Example 12

Physical blocking of the glass surface by a hydrophobic mask was used in this example to demonstrate the need for LPD solution contact with the glass surface for film formation to occur. A single gooseneck liner was immersed to a depth of 1 inch into melted paraffin wax and then removed. After the wax had hardened, the liner was immersed into an SSFA solution as in Example 3, but containing Rhodamine B as the dye. After the deposition treatment, the liner was rinsed with water, and then hot toluene. The pink clear coating was only present where the contact with the wax had not occurred, the latter having served as an effective mask against silica deposition.

### Example 13

Multiple thermochromic indicators in multiple deposition steps were used in this example. The partially coated liner from Example 12 was immersed in an SSFA solution containing methylene blue as in Example 3, but with about 10% of the dye concentration. The liner was then rinsed well with water and dried. The liner exhibited a blue color in the region where the wax mask had originally been, and a purple color where the Rhodamine B had already been deposited. It was then placed into an oven exposed to air and heated in increments of 50° C., holding for 30 minutes at each temperature. The first noticeable changes occurred at 200° C. The light blue liner had lost the Methylene Blue dye component, leaving behind a pink Rhodamine B color in the originally dyed region. The pink color remained until the temperature testing reached the 400° C. hold step, when it became clear and colorless. The stepwise color loss demonstrated an irreversible maximum temperature indicating thermochromic effect.

### Example 14

In another example of multiple thermochromic indicators, but co-deposited within the same film, a borosilicate glass single gooseneck liner was immersed in an SSFA solution containing a mixture of methylene blue and neutral red dyes as in Example 3. The liner was then rinsed well with water and dried. It was dark purple in color and smooth. It was placed in an oven and heated gradually to 400° C. using the process described in Example 13. At 200° C., the dark blue liner turned much darker, appearing almost black. The dark liner remained black until the hold at 350° C. where it turned dark brown. This corresponded to the decomposition of the heavily doped methylene blue dye leaving only the neutral red dye. On continued heating to 400° C., the liner turned a light brown color similar to that seen with the liner in Example 8. The stepwise color change in this Example thus demonstrated an irreversible maximum temperature indicating thermochromic effect.

### Example 15

The effect of deposition of silica using the process of this invention on surfaces other than silica was shown in this example. A single gooseneck liner was labeled with a silkscreened ceramic glaze decal and heated in a muffle furnace at 550° C. for several hours to fuse the pigment into the glass surface. After cooling, the liner was immersed into an SSFA solution as in Example 3 above, but with Rhodamine B as the dye. It exhibited a pink color. After rinsing and drying, the liner was examined for signs of etching or attack on the inorganic pigment. None was apparent, and the image resolution was not affected. The liner was now smooth over its entire surface, where the image had imparted a rough feel before. Microscopic examination revealed the presence of a smooth, conformal pink film over the entire article.

### Example 16

A single gooseneck borosilicate glass liner was loaded with 4 μm fused quartz wool and treated the same as the liner in Example 9, but with the omission of the Rhodamine B indicator. The treatment provided a very smooth and colorless liner. When tested chromatographically for inertness, this liner gave essentially identical results compared to the performance of the light pink colored liner produced in Example 9.

### Example 17

In this example, the interior of a sample vial is deactivated using one preferred embodiment of this invention. A 40 mL borosilicate glass screw cap vial was filled with 3 N hydrochloric acid and allowed to stand for 30 minutes. The solution was removed and the vial was rinsed with deionized water until neutral pH was obtained. Silica saturated fluorosilicic acid solution prepared in Example 1 was allowed to warm to room temperature and was held for 24 hours. The excess silica was removed by filtration and 20 mL of this solution was placed in the acid leached vial. The solution was diluted with 20 mL of water that had 2 drops of a 0.2% Rhodamine B solution added to it. The vial was capped, mixed well, and allowed to stand 6 hours with occasional swirling. The solution was then removed and the vial rinsed with water until the pH was neutral. A faint pink transparent coloration of the glass was observed.

The vial was placed in a stainless steel vessel, purged with helium, and heated to 250° C. for 5 hours to anneal the freshly deposited silica and reduce the overall fluoride content. The vial was then deactivated using standard reagents and processes. The light pink coating on the vial interior was very smooth and repellant to liquid water. Acetone was added to the midpoint of the vial, which was then capped. The solution showed no hint of dye leaching after 3 weeks exposure to the solvent.

The references referred to in this application and listed below are hereby incorporated herein by reference.

### REFERENCES

1. Konrad Grob in “Split and Splitless Injection for Quantitative Gas Chromatography, 4^{th} Ed., Wiley-VCH, 2001.
2. Anal. Chem. 2002, 74, 10-16 “The Two Options for Sample Evaporation in Hot GC Injectors: Thermospray and Band Formation. Optimization of Conditions and Injector Design” Koni Grob and Maurus Biedermann.
3. U.S. Pat. No. 5,954,862 “Sample Inlet Liner” William H. Wilson.
4. U.S. Pat. No. 5,472,670 “Gas Chromatography Sample Injector and Apparatus Using Same” Peter de B. Harrington and Hans P. Whittenherg.
5. U.S. Pat. No. 5,119,669 “Sleeve Units for Inlet Splitters of Capillary Gas Chromatographs” Paul H. Silvis.
6. U.S. Pat. No. 6,565,634 “Injection Liner” Wil van Egmond.
7. U.S. Pat. No. 6,719,826 “Method and Apparatus for Sample Injecting in Gas Chromatography” Ryoichi Sasano, Motoaki Satoh, and Yutaka Nakanishi.
8. U.S. Pat. No. 6,498,042 “PTFE Matrix in a Sample Inlet Liner and Method of Use” William H. Wilson.
9. U.S. Pat. No. 4,035,168 “Nonreactive Inlet Splitter for Gas Chromatography and Method” Walter G. Jennings.
10. U.S. Pat. No. 5,997,615 “Large-Sample Accessory for a Gas Chromatograph” Huan V. Luong, Hsing Kuang Lin, Howard Fruwirth, George S. Mueller.
11. U.S. Pat. No. 6,203,597 “Method and Apparatus for Mass Injection of Sample” Ryoichi Sasano, Kazuhiko Yamazaki, Masahiro Furuno.
12. U.S. Pat. No. 6,494,939 “Zero-Dilution Split Injector Liner Gas Chromatography” Andrew Tipler.
13. U.S. patent application Ser. No. 12/157,202, Paul H. Silvis and Thomas E. Kane, Restek Corporation, patent pending.
14 U.S. Patent Application Ser. No. 61/208,289, Brian A. Jones and Thomas E. Kane, Restek Corporation, patent pending.
15. “A Guide To Gas Chromatography”, W. Rodel and G. Wolm, Huthig Verlag, GmbH, Heidelberg, Germany.
16. For example, Catalog #s: 20726, 20974, 21700, and 21702 in the 2009 Restek Corporation Product Catalog.
17. Restek Corporation 2009 Catalog.
18. Journal of Non-Crystalline Solids 343 (2004) 3-12 “Leaching of Borosilicate Glasses. I. Experiments” A. Ledieu, F. Devreux, P. Barboux, L. Sicard, O, Spalla
19. Journal of Non-Crystalline Solids 343 (2004) 13-25 “Leaching of Borosilicate Glasses. II. Model and Monte-Carlo simulations” F. Devreux, A. Ledieu, P. Barboux, Y. Minet
20. U.S. Pat. No. 2,106,744 “Treated Borosilicate Glass” H. P. Hood and M. E. Nordberg
21. Physics and Chemistry of Glasses, 29(3), (1988), 106-120 “The Structure of Leached Sodium Borosilicate Glass” B. C. Bunker, t D. R. Tallan, T. J. Headley, G. L. Turner, R. J. Kirkpatrick
22. Journal of Materials Science 36 (2001) 1331-1341 “A Simplified Model for Glass Dissolution in Water” F. Devreux, Ph. Barboux, M. Filoche, B. Sapoval
23. Kinetics and Catalysis, 42(5), (2001), 693-702 “Catalysts Based on Fiberglass Supports: I. Physicochemical Properties of Silica Fiberglass Supports” L. G. Simonova, V. V. Barelko, 0. B. Lapina, E. A. Paukshtis, V. V. Terskildi, V. I. Zaikovskii, and B. S. Bal'zhinimaev
24. U.S. Pat. No. 6,511,760 “Method of Passivating a Gas Vessel or Component of a Gas Transfer System Using a Silicon Overlay Coating” Gary A: Barone, Andy S. Schuyler, and Joseph Stauffer.
25. U.S. Pat. No. 7,070,833 “Method for Chemical Vapor Deposition of Silicon on to Substrates for Use in Corrosive and Vacuum Environments” David A. Smith, Gary A. Barone, Martin E. Higgins, Bruce R. F. Kendall, and David J. Lavrich.
26. US Patent Appl. 2009/0029178 “Process for the Modification of Substrate Surfaces Through the Deposition of Amorphous Silicon Layers Followed by Surface Functionalization with Organic Molecules and Functionalized Structures” David A. Smith, Gary A. Barone, and Martin E. Higgins.
27. J. Mater. Chem., 2006, 16, 2890-2895 “Selective self-assembly of single walled carbon nanotubes in long steel tubing for chemical separations” Mahesh Karwa, Zafar Iqbal and Somenath Mitra
28. U.S. Pat. No. 4,468,420 “Method for Making A Silicon Dioxide Coating” Hideo Kawahara, Hirotsugu Nagayama, Hisao Honda
29. U.S. Pat. No. 2,490,662 “Skeletonizing Glass” S. M. Thomsen
30. J. Am. Chem. Soc. 72, 2798 “High-Silica Fluosilicic Acids” S. Milton Thomsen
31. J. Am. Chem. Soc. 74, 1952, 1690-1693 “High-Silica Fluosilicic Acids: Specific Reactions and the Equilibrium with Silica” S. Milton Thomsen
32. Monatshefie fur Chemie 99, (1968) 1177-1185 “Fluorkomplexe des Siliciums in Wiillriger Losung” K. Kleboth
33. U.S. Pat. No. 4,008,130 “Method for Distilling a Ternary Azeotrope of Fluorosilicic Acid, Hydrogen Fluoride and Water” Joel F. M. Leathers, Donald W. Calvin
34. J. Electrochem. Soc.: Solid-State Science And Technology 135(8),(1988) 2013-2016 “A New Process for Silica Coating” Hirotsugu Nagayama, Hisao Honda, and Hideo Kawahara
35. IEEE Symp. On VLSI Technology 1990 2-2 “A New Interlayer Formation Technology for Completely Planarized Multilevel Interconnection by Using LPD” T. Rotuma, T. Katoh, Y. Yamada, J. Shimizu, and Y. Murao
36. IEEE Symp. 1991 25.1.1 “A 7 Mask CMOS Technology Utilizing Liquid Phase Selective Oxide Deposition” K. Kanba, T. Horiuchi, T. Homma, Y. Murao and K. Okumura
37. J. Non-Crystalline Solids 151 (1992) 102-108 “Growth Mechanisms of Silica Glasses Using the Liquid Phase Deposition (LPD)” Koichi Awazu, Hiroshi Kawazoe and Kazuhiko Seki
38. U.S. Pat. No. 5,114,760 “Method for Manufacturing Layer Built Material with Silicon Dioxide Film Containing Organic Colorant and the Layer-Built Material Manufactured Thereby” Kazuo Takemura; Juichi Ino; Hideo Kawahara; Masaki Kitaoka
39. J. Electrochem. Soc., 140(8), (1993) 2410-2414 “A Selective Si02 Film-Formation Technology Using Liquid-Phase Deposition for Fully Planarized Multilevel Interconnections” Tetsuya Homma, Takuya Katoh, Yoshiaki Yamada, and Yukinobu Murao
40. U.S. Pat. No. 5,232,781 “Method for Manufacturing Layer-Built Material with Silicon Dioxide Film Containing Organic Colorant and the Layer-Built Material Manufactured Thereby” Kazuo Takemura; Juichi Imo; Hideo Kawahara; Masaki Kitaoka
41. J. Electrochem. Soc., 141(11), (1994) 3214-3218 “The Initial Growth Mechanism of Silicon Oxide by Liquid-Phase Deposition” Jenq-Shiuh Chou and Si-Chen Lee
42. Bull. Chem. Soc. Jpn., 68, (1995) 1275-1279 “The Absorption Spectra of pH Indicator Dyes Doped in Silica Thin Films Prepared by the Method of Liquid Phase Deposition and the Spectroscopic Estimation of Effective Acidity of Internal Hydroxyl Groups of Silica” Satoshi Suzuki, Naoaki Tokou, Toshiaki Mabuchi, Tsuyoshi Nakajima, Juichi Ino, Kazuo Takemura, and Hideo Kawahara
43. IEEE TRANSACTIONS on Electron Devices, 42(11), (1995) 1918 “Planarization of Amorphous Silicon Thin Film Transistors by Liquid Phase Deposition of Silicon Dioxide” Maw-Song Chen, Jenq-Shiuh Chou, and Si-Chen Lee
44. J. Non-Crystalline Solids 187 (1995) 81-85 “Effects of Plasma Treatment on the Properties of Room-Temperature Liquid-Phase Deposited (LPD) Oxide Films” Ching-Fa Yeh, Shyue-Shyh Lin
45. J Electrochem. Soc., 144(3), (1997) 1144-1149 “On Liquid-Phase Deposition of Silicon Dioxide by Boric Acid Addition” Peng-Heng Chang, Chen-Tang Huang, and Jin-Shown Shie”
46. J. Appl. Phys. 82(11), (1997), 5788 “Extremely Low Temperature Formation of Silicon Dioxide on Gallium Arsenide” M. P. Houng, C. J. Huang, Y. H. Wang, N. F. Wang, and W. J. Chang
47. IEEE Transactions On Instrumentation And Measurement, 47(3), (1998), 698 “Optical Properties of Fluorinated Silicon Oxide Films by Liquid Phase Deposition for Optical Waveguides” Tetsuya Homma, Atsushi Satoh, Seiji Okada, Masahiro Itoh, Masaki Yamaguchi, and Hideo Takahashi
48. J. Vac. Sci. Technol. A, 16(4), (1998) 2646 “Improved Formation of Silicon Dioxide Films in Liquid Phase Deposition” C. J. Huang, M. P. Houng, Y. H. Wang, and N. F. Wang, Jiann-Ruey Chen
49. U.S. Pat. No. 5,753,311 “Method for Forming Oxide Layer” Ming-Kwei Lee
50. J. Vac. Sci. Technol. A 17(1), (1999), 102 “Deposition of High Quality Silicon Dioxide on Hg₁₂ₓCdₓTe by Low-Temperature Liquid Phase Deposition Method” N. F. Wang, W. J. Chang, M. P. Houng, Y. H. Wang, and C. J. Huang
51. Semicond. Sci. Technol. 18 (2003) L45-L48 “Selective and Blanket Liquid Phase Deposition of Silicon Dioxide on Silicon Nitride and Tungsten Silicide” Ming-Kwei Lee, Chung-Min Shih and Chi-Da Yang
52, IEEE 1-4244-0161-5/06 “Plasma Treatment on the Plastic Substrates for Liquid Phase Deposited SiO₂ Films for Flexible Electronics Applications” Yu-Ju Lin, Chih-Chieh Yeh, Li-Ming Huang, Ten-Chin Wen and Yeong-Her Wang
53. Phys. Stat. Sol. (a) 205(2), (2008) 231-234 “Liquid Phase Deposited Silicon Oxide with Lower Boron Impurity Grown on Gallium Nitride by Temperature-Difference Method” Ming-Kwei Lee, Chen-Lin Ho, and Jia-Yi Zeng
54. J. Ceramic Soc. Jpn. 117(3), (2009) 326-329 “Organic Compound/Metal Oxide Hybrid Thin Film Prepared by the Liquid Phase Deposition Method” Shigehito Deki, Yasuaki Kodama and Minoru Mizuhata
55. J. Ceramic Soc. Jpn 117(3), (2009) 335-339 “Equilibrium of Dissolving Species in Aqueous H₂SiF₆ Solution during the Liquid Phase Deposition Reaction” Minoru Mizuhata, Yasuhiro Saito, Masao Takee and Shigehito Deki
56. U.S. Pat. No. 4,693,916 “Method of Depositing a Silicon Dioxide Film” Hirotsugu Nagayama; Hisao Honda; Hideo Kawahara
57. Appl. Surf. Sci. 48/49 (1991) 405-408 “Formation of Silicon Dioxide Films in Acidic Solutions” Akihiro Hishinuma, Takuji Goda, Masaki Kitaoka, Shigeo Hayashi and Hideo Kawahara
58. J. Soc. Mat. Sci., Japan, 43(494), (1994), 1437-1443 “Formation of SiO₂ Thin Film on SUS304 Stainless Steel by Liquid Phase Deposition” Seiji Nitta and Yuji Kimura
59. U.S. Pat. No. 5,326,720 “Method for Producing Silicon Dioxide Film Which Prevents Escape of Si Component to the Environment” Takuji Goda; Yasuto Sakai; Akihiro Hishinuma; Hideo Kawahara; Shigehito Deki
60. U.S. Pat. No. 5,395,645 “Method for Forming a Silicon Oxide Film on a Silicon Waffer” Masako Kodera; Masami Watase; Shiro Mishima; Katsuya Okumura
61. Thin Solid Films 392 (2001) 294-298 “Silica Coating on Plastics by Liquid Phase Deposition by LPD Method” Yasuto Sakai, Hodaka Norimatsu, Yasuhiro Saito, Hiroyuki Inomata, Toshiaki Mizuno
62. SPIE Vol. 1513 Glasses for Optoelectronics 11, (1991) 198-203 “Preparation of SiO₂ Film Utilizing Equilibrium Reaction in Aqueous Solution” H. Kawahara, Y. Sakai, T. Goda, A. Hishinuma and K. Takemura
63. U.S. Pat. No. 5,073,408 “Method of Depositing a Silicon Dioxide Film” Takuji Godaj Hirotsugu Nagayama, Hideo Kawahara,
64. U.S. Pat. No. 5,132,140 “Process for Depositing Silicon Dioxide Films” Takuji Goda; Hirotsugu Nagayama; Hideo Kawahara; Yasuto Sakai; Akihiro Hishinuma
65. Mat. Res. Soc. Symp. Proc. 511, (1998), 57 “Newly Developed Low-K and Low-Stress Fluorinated Silicon Oxide Utilizing Temperature-Difference Liquid-Phase Deposition Technology” Ching-Fa Yeh, Yueh-ChuanLee, and Su-Chen Lee
66. J. Electrochem. Soc., 147(1) (2000) 330-334 “Comprehensive Investigation on Fluorosilicate Glass Prepared by Temperature-Difference-Based Liquid-Phase Deposition” Ching-Fa Yeh, Yueh-Chuan Lee, Kwo-Hau Wu, Yuh-Ching Su, and Su-Chen Lee
67. J Electrochem. Soc, 147 (11) 4268-4272 (2000) “Reliability of Fluorinated Silicon Oxide Film Prepared by Temperature Difference-Based Liquid Phase Deposition” Ching-Fa Yeh, Yueh-Chuan Lee, and Su-Chen Lee
68. Appl. Phys. Lett. 64(15), (1994), 1971 “Improved Process for Liquid Phase Deposition of Silicon Dioxide” Jenq-Shiuh Chou and Si-Chen Lee
69. EDMS'94 11-21-78 “High Quality Thin Oxide Films Formed by Using Ultra Low Temperature Liquid Phase Deposition” Ching-Fa Yeh, Shyue-Shyh Lin and Tzy-Yan Hong
70. EDMS'94 I-3-10 “Radiation Hardness of Fluorinated Oxides Prepared by Liquid Phase Deposition Method Following Rapid Thermal Oxidation” Wei-Shin Lu, JenqShiuh Chou, Si-Chen Lee, and Jenn-Gwo Hwu
71. J. Electrochem. Soc., 141(11), (1994), 3177-3181 “The Physicochemical Properties and Growth Mechanism of Oxide (SiO₂₋ₓFₓ) by Liquid Phase Deposition with H₂O Addition Only” Ching-Fa Yeh, Chun-Lin Chen, and Guan-Hong Lin
72. Appl. Phys. Lett., 66(8), (1995), 938 “Bond-Structure Changes of Liquid Phase Deposited Oxide (SiO₂₋ₓFₓ) on N₂ Annealing” Ching-Fa Yeh and Chun-Lin Chen, Water Lur and Po-Wen Yen
73. Microelectronic Eng. 28 (1995) 101-104 “Fabrication of MOSFETs Using Low-Temperature Liquid-Phase Deposited Oxide” Ching-Fa Yeh, Shyue-Shyh Lin and Tzy-Yan Hong
74. J. Electmchem. Soc., 142(10), (1995), 3579-3583 “Controlling Fluorine Concentration and Themmal Annealing Effect On Liquid-Phase Deposited SiO₂₋ₓFₓ Films” Ching-Fa Yeh and Chun-Lin Chen
75. IEEE Transactions on Electron Devices, 43(4), (1996), 599 “Application of Liquid Phase Deposited Silicon Dioxide to Metal-Oxide-Semiconductor Capacitor and Amorphous Silicon Thin-Film Transistor” Jenq-Shiuh Chou and Si-Chen Lee
76. IEEE Electron Device Letters, 17(4), (1996), 172 “Reliable Fluorinated Thin Gate Oxides Prepared by Liquid Phase Deposition Following Rapid Thermal Process” Wei-Shin Lu and Jenn-Gwo Hwu
77. J. Electrochem. Soc., 143(8), (1996), 2658-2662 “Stress in Liquid-Phase Deposited Oxide Films” Ching-Fa Yeh, Shyue-Shyh Lin, and Water Lur
78, U.S. Pat. No. 5,506,006 “Process for Depositing Silicon Dioxide by Liquid Phase Diposition” Jeng-Shia Chou; Si-Chen Lee
79. Jpn. J. Appl. Phys. 38 (1999), 5715-5719 “Silicon Dioxide Film with Low Dielectric Constants using Liquid-Phase Deposition” Phonekeo Chanthamaly, Taro Arakawa And Nobuo Haneji
80. Mat. Res. Soc. Symp. Proc. 716, (2002), B7.9.1 “Device Quality SiO₂ Films by Liquid Phase Deposition (LPD) at 48° C.” M. Manhas, T. J. Pease, R. Cross, S. C. Bose, D. P. Oxley, M. M. De Souza, E. M. Sankara Narayanan
81. Appl. Phys. Lett., 82(4), (2003), 589 “Strain-Induced Growth of SiO₂ Dots by Liquid Phase Deposition” C. W. Liu, B.-C. Hsu, K.-F. Chen, M. H. Lee, and C.-R. Shie, Pang-Shin Chen
82. Nano Letters, 3(6), (2003), 775-778 “Silica Coated Single Walled Carbon Nanotubes” Elizabeth A. Whitsitt and Andrew R. Barron
83. Rice University Thesis, December 2004 “Liquid Phase Deposition of Silica: Thin Films, Colloids and Fullerenes” Elizabeth A. Whitsitt
84. J. Mater. Sci. 42, (2007), 7381-7388 “Silica Coating of Vapor Grown Carbon Fibers” Huma R. Jafry, Elizabeth Whitsitt, Andrew R. Barron
85. Mater. Res. Soc. Symp. Proc. 965, (2007) 0965-S06-01 “Fabrication of Organic Thin Film Transistors using Low Temperature, Soluble Silicon Oxide as the Gate Dielectrics” Jeng-Hua Wei, HorngJiunn Lin, and Ying-Ren Chen
86. Molecular Crystals and Liquid Crystals, 267, (1995), 169-174 “Organic Dye-Doped Silica Films by the Liquid Phase Deposition Method as Potential New Hybrid Materials for Photonics” Toshihiko Nagamura; Hisahiro Shimizu
87. Molecular Crystals and Liquid Crystals, 277, (1996), 215-226 “The Thermochromic Behavior of Cyanine Dyes Doped in Silica Phases Prepared by Liquid-Phase-Deposition Method” Takashi Usui; Satoshi Suzuki; Masanobu Kojima; Tsuyoshi Nakajima; Juichi Ino; Kazuo Takemura
88. Molecular Crystals and Liquid Crystals, 316, (1998), 119-122 “Spectroscopic Properties of Porphyrin-doped Silica Films by the Liquid Phase Deposition Method” Toshihiko Nagamura; Hirohisa Smmizu; Hiroyasu Ohkoso
89. Nippon Kagakkai Koen Yokoshu, 79(1), (2001), 289 “Preparation and Characterization of Organic Dye/Various Metal Oxides Composite Thin Film Prepared by the Liquid Phase Deposition (LPD) Method” Deki Shigehito, Fujii Shoji, Akamatsu Kensuke, Mizuhata Minoru, Kajinatni Akihiko
90. J. Cer. Soc. Jpn., 117(3), (2009), 228-235 “Recent Progress in the Synthesis of Oxide Films from Liquid Solutions” Harshil Parikh and Mark R. De Guire
91. U.S. Pat. No. 5,470,681 “Phase Shift Mask using Liquid Phase Oxide Deposition” Timothy A. Brunner; Derek B. Dove; Louis L. Hsu
92. Solar Energy Materials 8 (1983) 399-409 “Transparent High Surface Area Porous Supports as New Materials for Luminescent Solar Concentrators” R. Reisfeld, N. Manor, and D. Avnir.
93. Spectroscopy Lett. 21(2) 1988 127-145 “Vycor Porous Glass (Thirsty Glass) as a Reaction Medium for Optical Waveguide Based Chemical Vapor Detectors” T. J. Novak and R. A. Mackay.
94. Journal of Non-Crystalline Solids 319 (2003) 163-173 “Effect of the Photodeposition of Tin on the Consolidation of Porous Vycor Glass” Jinquan Dong, Dehipawalage Sunil, Harry D. Gafney, Steven A. Schwarz.
95. Journal of Non-Crystalline Solids 74 (1985) 395-406 “Organic Fluorescent Dyes Trapped In Silica And Silica-Titania Thin Films By The Sol-Gel Method. Photophysical, Film And Cage Properties” David Avnir, Vered R. Kaufman And Renata Reisfeld.
96. Appl. Phys. Lett. 67, 3703 (1995) “Two-photon Pumped Cavity Lasing in Novel Dye Doped Bulk Matrix Rods” Guang S. He, Chan F. Zhao, Jayant D. Bhawalkar, and Paras N. Prasad.
97. Physical Review Letters, Volume 56, Issue 2, Jan. 13, 1986, pp. 197-200 “Self-Diffusion of a Molecule in Porous Vycor Glass” W. D. Dozier, J. M. Drake, and J. Klafter,
98. U.S. Pat. No. 7,364,700 “Ozone Gas Sensing Element Detection Apparatus, and Measurement Method” Yasuko Marne, Shigeo Ogawa, and Seizou Sakata, Tohru Tanaka.
99. U.S. Pat. No. 6,402,037 “Two-Photon Upconverting Dyes and Applications” Paras N. Prasad, Jayant D. Bhawalker, Ping Chin Cheng, Shan Jen Pan.
100. US Patent Appl. 2006/0194122 “Hologram Recording Material and Optical Recording Medium” Hiroo Takizawa.
101. U.S. Pat. No. 7,232,637 “Light Sensitive Media for Optical Devices Using Organic Mesophasic Materials” Michael C. Cole, Timothy J. Trentler.
102. Microporous and Mesoporous Materials 60 (2003) 19-30 “Porous glasses in the 21st century—a short review” D. Enke, F. Janowski, W. Schwieger.
103. U.S. Pat. No. 4,878,224 “Dye Lasers” James E. Kuder, James L. McGinnis, Harris A. Goldberg, Timothy R. Hart, Tessie M. Che.
104. Journal of Sol-Gel Science and Technology, 2, 81-86 (1994) “Structures and Properties of Ormosils” John D. Mackenzie.
105. Khimiya Geterotsiklicheskikh Soedinenii, No. 4, pp. 435-459, April, 1979. “Synthesis and Properties of Spiropyrans That are Capable of Reversible Opening of the Pyran Ring (Review)” E. R. Zakhs, V. M. Martynova, and L. S. Efros.
106. Acc. Chem. Res., 1995, 28 (8), 328-334 “Organic Chemistry within Ceramic Matrixes: Doped Sol-Gel Materials” David Avnir.
107. Applied Spectroscopy, 53(7), 1999 785-791 “Reduction of Indicator Leaching from Doped Sol-Gels by Attachment of Macromolecular Carriers” Peter J. Skrdla, S. Scott Saavedra, and Neal R. Armstrong.
108. Journal of Non-Crystalline Solids 113 (1989) 137-145 “Applications Of The Sol-Gel Process For The Preparation Of Photochromic Information-Recording Materials: Synthesis, Properties, Mechanisms” David Levy, Shlomo Einhorn and David Avnir.
109. WO 2008/028128 “LIGAND EXCHANGE THERMOCHROMIC, (LETC), SYSTEMS” Vander Griend, Douglas, A.; Ogburn, Paul, H., Jr.; Millett, Frederick, A.; Millett, Frederick, C.; Winkle, Derick, D.; Byker, Harlan, J.; Veldkamp, Brad, S.
110. Chem. Mater., 1997, 9 (12), 2666-2670 “Photochromic Sol-Gel Materials” David Levy.
111. “Chromic Phenomena-Technological Applications of Colour Chemistry” ed. P. Bamfield, Published by The Royal Society of Chemistry, Thomas Graham House, Science Park, Milton Road, Cambridge.
112. U.S. Pat. No. 4,344,909 “Thermochromic Composition” F. J. A. M. C. De Blauwe,
113. Chem. Rev., 1968, 68 (6), 649-657 “Thermochromism of Inorganic Compounds” Jesse H. Day.
114. U.S. Pat. No. 6,890,377 “Thermochromatic Rylene Dyes” A. Bohm, M. Krieger, S. Becker, K. Mullen.
115. Chem. Rev., 2004, 104 (5), 2751-2776 “Photo-, Thermo-, Solvato-, and Electrochromic Spiroheterocyclic Compounds” Vladimir I. Minkin.
116. U.S. Pat. No. 7,304,008 “Thermochromic Material” A. V. Belykh, A. M. Efremov, M. D. Mikhailov.
117. Chem. Rev., 2000, 100 (5), 1817-1846 “Linear and Nonlinear Optical Properties of Photochromic Molecules and Materials” Jacques A. Delaire, and Keitaro Nakatani.

What is claimed is:

1. A container used for chromatographic sample analysis comprising a silica uppermost layer where the uppermost layer is deposited using a liquid phase deposition process.

2. The container of claim 1 where the uppermost layer has % wt fluorine content greater than zero.

3. The container in claim 1 where the silica uppermost layer is deactivated with silanes.

4. A container used for chromatographic sample analysis comprising
(a) a silica uppermost layer where the uppermost layer is deposited using a liquid phase deposition process, and
(b) a glass substrate having a softening point less than 1600° C.

5. The container of claim 4 where the uppermost layer has % wt fluorine content greater than zero.

6. The container in claim 4 where the silica uppermost layer is deactivated with silanes.

7. A container used for chromatographic sample analysis comprising
(a) at least one silica uppermost layer where the uppermost layer is deposited using a liquid phase deposition process, and
(b) at least one indicator being present in at least one of the silica uppermost surface layers.

8. The container of claim 7 where the indicator change is exhibited as a spectral shift or change in light absorbance in at least part of the container, the indicator response being due to a change in the temperature of the inlet container.

9. The container of claim 7 where the indicator change is exhibited as a change in the degree of transmittance of light through the glass in at least part of the container, the indicator response being due to a change in the temperature of the container.

10. The container of claim 7 where the indicator change is exhibited as a change from at least one color to a darker one in at least part of the container, the indicator response being due to an increase in the temperature of the container.

11. The container of claim 7 where the indicator is based on a fluorescence signal.

12. The container of claim 7 where the indicator change is reversible with the change in temperature.

13. The container of claim 7 where the indicator change is irreversible with the change in temperature.

14. A method for determining the temperature of a container, comprising the steps of
providing a container having at least one silica uppermost layer, where the at least one silica uppermost layer is deposited using a liquid phase deposition process, and at least one indicator present within at least part of at least one of the silica uppermost layer, where the at least one indicator is thermoehromic, and
visually identifying the temperature of the container by observing coloration of the container, the temperature of the container being correlative with coloration of the container.

15. A method for determining duration of use of a container, comprising the steps of
providing a container having at least one silica uppermost layer, where the at least one silica uppermost layer is deposited using a liquid phase deposition process, and at least one indicator present within at least part of at least one of the silica uppermost layer, where the at least one indicator is thermochromie and has an irreversible response, and
visually identifying how much the container has been used by observing coloration of the container, the amount of use of the container being correlative with coloration of the container.

16. A method for determining proper orientation of a container, comprising the steps of
providing a container having at least one silica uppermost layer, where the at least one silica uppermost layer is deposited using a liquid phase deposition process, and at least one indicator present within a part of at least one of the silica uppermost layer, and
visually identifying the location on the container of the part of the at least one of the silica uppermost layer having the at least one indicator, the location on the container of the part of the at least one of the silica uppermost layer having the at least one indicator being correlative with how the container is oriented during use.

17. A method for distinguishing a first container from a second container, comprising the steps of
providing a first container having at least one silica uppermost layer, where the at least one silica uppermost layer is deposited using a liquid phase deposition process, and at least one indicator present within at least part of at least one of the silica uppermost layer, and
comparing a second container with the first container to determine if the second container has an indicator that matches the indicator of the first container.

18. A method for permanently introducing at least one indicator to at least part of at least one of the silica uppermost layers of the container, comprising the steps of
providing a container used for chromatography sample analysis, and
depositing at least one silica uppermost layer on the container using a liquid phase deposition process,
at least part of the at least one silica uppermost layer having at least one indicator present therein.

19. The method of claim 18 where the indicator is thermochromic.

20. The method of claim 18 where the indicator is fluorescent.

21. The method of claim 18 where the indicator is permanently colored and remains unchanged through multiple thermal cycles.

22. The method of claim 18 where the substrate is deactivated with silanes.

23. The method of claim 18 where the indicator color intensity decays over time in response to thermal or oxidative stresses.

24. The method of claim 18 where the indicator is photochromic.

25. The method of claim 24 where the photochromic indicator is converted to a colored form through exposure to light of a particular frequency range.

26. The method of claim 24, where the conversion to a colored form is in selected regions of the substrate, through photolithographic or focused exposure means.

27. The method of claim 24 where the photochromic effect is thermally erasable.

28. The method of claim 18 where the indicator is only visible under ultraviolet light.

29. A method of providing a smooth surface to a container used for chromatographic sample analysis, comprising the steps of
providing a container used for chromatographic sample analysis, and
depositing at least one silica uppermost layer on the container using a liquid phase deposition process.
